(19) United States
(12) Patent Application Publication
(10) Pub. No.: US 2025/0099256 A1
(43) Pub. Date: Mar. 27, 2025

(54) DEVICES AND METHODS FOR INTERPHALANGEAL JOINT FUSION

(71) Applicant: Intelivation Technologies LLC, Saint Simons Island, GA (US)

(72) Inventors: David Rathbun, Saint Simons Island, GA (US); Amit Sinha, Saint Simons Island, GA (US); David Posillico, Saint Simons Island, GA (US); David Murphy, Saint Simons Island, GA (US)

(21) Appl. No.: 18/892,002

(22) Filed: Sep. 20, 2024

Related U.S. Application Data
(60) Provisional application No. 63/584,613, filed on Sep. 22, 2023.

(51) Int. Cl. A61F 2/42; A61F 2/30

(52) U.S. Cl. CPC A61F 2/4241; A61F 2002/30115; A61F 2002/30622; A61F 2002/4243

(57) ABSTRACT

System and methods for bone fusion are disclosed. The systems and method are directed to forming complementary surfaces on bones on either side of an interphalangeal joint and limiting the relative movement thereof so as to promote fusion of the joint.

### CROSS-REFERENCE TO RELATED APPLICATION

This application claims the benefit of U.S. Provisional Application No. 63/584,613 filed, Sep. 22, 2023, entitled “Devices and Methods for Surgical Treatment of Hammerdesis,” the contents of which are incorporated herein by reference in its entirety.

### TECHNICAL FIELD

The present application is directed to medical devices and methods for correcting interphalangeal deformities and facilitating joint fusion in a patient.

### BACKGROUND

Interphalangeal joint deformities and injuries can cause severe pain, lead to instability of the joint, and impair proper motion and function. Such ailments commonly affect proximal interphalangeal (PIP) joints of the hand and foot. For example, hammertoe is a common foot ailment that affects millions of individuals worldwide. It can be attributed to a combination of genetic predisposition, footwear choices, and underlying medical conditions. The condition is characterized by the abnormal bending of the proximal interphalangeal joint of one or more toes, causing the affected toes to resemble a hammer. This condition can result in discomfort, pain, and impaired mobility, significantly impacting an individual's quality of life. In particular, hammer toe often results in pain and discomfort, especially when wearing shoes that do not accommodate the deformity. The pressure and friction on the bent joint can cause corns, calluses, and blisters. As the condition progresses, the affected toes may become rigid, making it difficult to move or flex them properly. This limited mobility can affect an individual's balance and gait. In addition to functional issues, hammer toe can also be a source of embarrassment and self-consciousness due to the visible deformity of the toes. Untreated hammer toe can lead to more severe complications, such as ulcers, infection, and even joint contractures, which further exacerbate the problem.

Various treatment options are available for hammer toe, ranging from conservative measures to surgical interventions. Some of the commonly employed methods include footwear modifications and orthotic devices. Patients are often advised to wear shoes with a roomy toe box and adequate arch support to alleviate pressure on the affected toes. Custom orthotic inserts can help to redistribute pressure and provide support to the toes, potentially slowing the progression of the deformity. Physical therapy exercises may be recommended to improve joint flexibility and muscle strength in the toes. In some instances, medications such as nonsteroidal anti-inflammatory drugs (NSAIDs) or corticosteroid injections may be used to manage pain and inflammation associated with hammer toe. For some patients, splints and braces can be worn to help straighten the affected toes and prevent them from becoming more rigid. In cases where conservative methods prove ineffective, surgical procedures may be necessary. These can include joint repositioning, fusion, or correction of soft tissue abnormalities. Among other challenges, effective joint/bone fusion entails careful preparation of complementary bone surfaces fixing the relative positions of the bones to be fused.

Despite the availability of these treatment options, there remains a need for improved and innovative methods and devices for the treatment of hammer toe. The present disclosure addresses these needs by providing an efficient and effective approach to the management and correction of hammer toe deformities. The systems and methods described in the present disclosure improve patient outcomes and reduce treatment and recovery times. Specifically, the systems and methods disclosed herein relate to forming complementary surfaces on the bones on either side of an interphalangeal joint and limiting the relative movement thereof so as to promote fusion of the joint.

### SUMMARY

Aspects of the present interphalangeal joint fusion system can reduce pain and correct deformities in an interphalangeal joint by facilitating fusion of the joint. Some aspects relate to methods and devices for forming complementary surfaces on the bones on either side of an interphalangeal joint such that the bones are better configured to mate and fuse. An additional aspect of the present interphalangeal joint fusion system limits relative movement (e.g., axial and rotational movement) of the bones on either side of the interphalangeal joint. Additional aspects of the present interphalangeal joint fusion system can facilitate anchoring of an implant within the interphalangeal joint. Furthermore, certain implementations can supplement patient anatomy in the event that bones on either side of the interphalangeal joint are not suitable for shaping.

In one implementation, the systems and methods described herein relate to a system including an implant. The implant includes an annular body having a proximal spike extending axially from a proximal surface of the annular body and a distal spike extending axially from a distal surface of the annular body. In some implementations, the implant is a counter-rotation ring. The system may be used in the treatment of hammertoe.

In some implementations, the proximal spike may include a plurality of proximal spikes extending axially from the proximal surface of the annular body and the distal spike may include a plurality of distal spikes extending axially from the distal surface of the annular body. In some implementations, the plurality of proximal spikes may be arranged circumferentially about the proximal surface of the annular body, and the plurality of distal spikes may be arranged circumferentially about the distal surface of the annular body. In some implementations, the plurality of proximal spikes and the plurality of distal spikes may be evenly spaced about the corresponding proximal surface and distal surface. In some implementations, a circumferential location of each of the plurality of proximal spikes on the proximal suface may correspond with a circumferential location of a corresponding one of the plurality of distal spikes on the distal surface. The axial spikes prevent relative rotational movement of two bones to be fused.

In some implementations, at least one of the proximal spike or the distal spike may have a decreasing width from the corresponding proximal surface or distal surface of the annular body toward an axial spike tip of the proximal spike or the distal spike. In some implementations, the side edges of the spike may define an increasing tapered surface from the spike tip to the corresponding proximal surface/distal surface. The axial spike tips may prevent relative axial movement of two bones to be fused.

In some implementations, at least one of the proximal spike or distal spike may include an inner surface that is angled radially inward toward a central axis of the annular body. In some implementations, the angled inner surface helps to position and/or advance the implant into the bone material. In some implementations, at least one of the proximal spike or distal spike may include a barb extending inward from an inner surface of the spike toward a central axis of the annular body. In some implementations, the barb may help prevent pullout/withdraw of the implant from the bone material.

In some implementations, at least one of the proximal spike or distal spike may include an outer surface extending generally parallel with a central axis of the annular body. In some implementations, at least one of the proximal spike or distal spike may include an inner surface extending generally parallel with a central axis of the annular body, and an outer surface having a decreasing taper from the corresponding proximal surface or distal surface of the annular body toward a spike tip of the proximal spike or distal spike.

In some implementations, the techniques described herein relate to a system, wherein at least one of the proximal spike or distal spike includes a barb extending outward from an outer surface of the spike away from a central axis of the annular body.

In some implementations, the annular body may include a plurality of circumferential slots arranged circumferentially about the annular body and extending at least partially through the annular body. In some implementations, at least one of the plurality of circumferential slots may extend through the annular body from the proximal surface to the distal surface. In some implementations, the plurality of circumferential slots may be spaced evenly about a circumference of the annular body. In some implementations, the plurality of circumferential slots may include an elongated body portion having a curvature corresponding to a curvature of the annular body.

In some implementations, the annular body may include a suture opening extending through the annular body.

In some implementations, the annular body may include a staple cutout extending radially inward from an outer surface of the annular body toward a central axis of the annular body.

In some implementations, the annular body may include a central lumen extending therethrough, an inner surface of the central lumen including a radial projection extending radially away from a central axis of the annular body. The radial projection may be referred to as a bear ear.

In some implementations, the annular body may include a central lumen extending therethrough. In some implementations, the central lumen may have an inner diameter ranging from 4.0 mm to 5.0 mm, and the annular member has an outer diameter ranging from 6 mm to 7 mm. In some implementations, the inner diameter of the central lumen may be about 4 mm. In some examples, the outer diameter of the annular member may be about 6.5 mm.

In some implementations, the annular body may have a thickness ranging from 0.5 mm to 1.5 mm. In some implementations, the annular body may have a thickness of about 1 mm.

In some implementations, the annular body may have a generally circular-shaped outer surface and a generally rectangular-shaped cross-section. In some implementations, the annular body may have a rectangular cross-section of about 3.63 mm\hat{ }2.

In some implementations, the system may further include a peg reamer. The peg reamer has a cannulated shaft defining a central lumen extending therethrough. The peg reamer further includes a cutting end provided at a first end of the cannulated shaft and a locking end provided at an opposing second end of the cannulated shaft from the cutting end. The cutting end includes a plurality of cutting arms extending from the cannulated shaft. Each cutting arm includes a cutting edge extending along an inner surface of each cutting arm.

In some implementations, the central lumen of the peg reamer cannulated shaft may define a central axis of the peg reamer, where and each of the cutting edges are spaced apart from the central axis of the cannulated shaft.

In some implementations, a spacing between each of the cutting edges and the central axis of the peg reamer cannulated shaft may range from 2.25 mm to 2.75 mm. In some implementations, the spacing between each of the cutting edges and the central axis may be about 2.56 mm.

In some implementations, the plurality of cutting arms may include four cutting arms. In some implementations, the plurality of cutting arms may be circumferentially arranged about the cutting end of the peg reamer cannulated shaft. In some implementations, the plurality of cutting arms may be arranged circumferentially around the central axis of the peg reamer. In some implementations, the plurality of cutting arms may be evenly spaced around the central axis.

In some implementations, rotation of the plurality of cutting arms about a central axis of the peg reamer may define a peg cutout region therebetween. In some implementations, the size and shape of the peg cutout region may correspond to the size and shape of the bone material removed from the patient's bone.

In some implementations, the peg cutout region may have a depth ranging from 4.5 mm to 5.5 mm. In some implementations, the depth of the peg cutout region may be about 5 mm. In some implementations, the techniques described herein relate to a system, wherein the peg cutout region has a first diameter adjacent a proximal end of the peg cutout region and a larger second diameter adjacent a distal end of the peg cutout region. In some implementations, the second diameter ranges from 5.0 mm to 5.25 mm. In some examples, the second diameter is about 5.12 mm. In some implementations, the cutting edges may slope outward relative to a central axis of the peg reamer toward a distal end of the cutting arms, wherein the peg cutout region second diameter is larger than the peg cutout region first diameter such that the peg cutout region is tapered.

In some implementations, the central lumen of the peg reamer may be sized and configured to receive a guide pin. In some implementations, the central lumen of the peg reamer may have a diameter ranging from 1.5 mm to 2.0 mm. In some implementations, the diameter of the central lumen may be about 1.8 mm.

In some implementations, the locking end of the peg reamer may include a flat extending axially along the cannulated shaft.

In some implementations, the locking end of the peg reamer may include a groove extending circumferentially around the cannulated shaft.

In some implementations, the system may further include a hole reamer. The hole reamer includes a cannulated shaft defining a central lumen extending therethrough. The hole reamer has a cutting end provided at a first end of the cannulated shaft and a locking end provided at an opposing second end of the cannulated shaft from the cutting end. The cutting end includes a plurality of cutting edges extending along an outer surface of the cutting end and a counterbore offset a distance from a distal end of the cutting end.

In some implementations, the central lumen of the hole reamer cannulated shaft may define a central axis of the hole reamer, wherein rotation of the plurality of cutting edges about the hole reamer central axis defines a hole cutout region. In some implementations, the size and shape of the hole cutout region may correspond to the size and shape of the bone material removed from/cavity created in the patient's bone.

In some implementations, the hole cutout region may have a depth ranging from 4.5 mm to 5.0 mm. In some implementations, the depth of the hole cutout region may be about 4.8 mm.

In some implementations, the hole cutout region may have a first diameter adjacent the counterbore and a second diameter adjacent the distal end of the hole reamer. In some implementations, the second diameter may range from 4.75 mm to 5.25 mm. In some examples, the second diameter is about 5.1 mm.

In some implementations, the plurality of cutting edges may slope inward relative to the central axis from the counterbore toward the distal end of the hole reamer such that the hole cutout region second diameter is larger than the hole cutout region first diameter and the hole cutout region is tapered toward the distal end.

In some implementations, the central lumen of the hole reamer may be sized and configured to receive a guide pin.

In some implementations, the central lumen of the hole reamer cannulated shaft may have a diameter ranging from 1.5 mm to 2.0 mm. In some implementations, the central lumen may have a diameter of about 1.8 mm.

In some implementations, the locking end of the hole reamer cannulated shaft may include a flat.

In some implementations, the locking end of the hole reamer cannulated shaft may include a groove.

In some implementations, the system may further a rescue dowel. The rescue dowel includes a tubular body defining a central lumen extending therethrough. The tubular body has a proximal end defining a proximal end opening extending to the central lumen and a distal end defining a distal end opening extending to the central lumen.

In some implementations, the proximal end and distal end may be sized and configured to frictionally engage with the hole cutout region. In some implementations, the tubular body may further define a plurality of axial slots arranged circumferentially about the tubular body and extending toward the central lumen. In some implementations, the plurality of axial slots may be spaced evenly about a circumference of the tubular body. In some implementations, the central lumen of the rescue dowel may be sized and configured to receive a guide pin.

In some implementations, the system may further include a guide pin. The guide pin is sized and configured to extend from and frictionally engage with a distal bone of a joint (e.g., a distal phalange) through at least a portion of a proximal bone of the joint (e.g., a proximal phalange). The guide pin is further sized and configured to be received within the central lumen of the peg reamer and the central lumen of the hole reamer. In some implementations, the guide pin may be sized and configured to be received within the central lumen.

In some implementations, the techniques described herein relate to a kit for the surgical treatment of deformity of the interphalangeal joints (e.g., hammertoe). The kit includes: an implant including an annular body including a proximal spike extending axially from a proximal surface of the annular body and a distal spike extending axially from a distal end of the annular body; a peg reamer including: a cannulated shaft defining a central lumen extending therethrough; a cutting end provided at a first end of the cannulated shaft, the cutting end including a plurality of cutting arms extending from the cannulated shaft, each of the plurality of cutting arms includes a cutting edge extending along an inner surface of the corresponding cutting arm; and a locking end provided at an opposing second send of the cannulated shaft from the cutting end; a hole reamer including: a cannulated shaft defining a central lumen extending therethrough; a cutting end provided at a first end of the cannulated shaft, the cutting end including a plurality of cutting edges extending along an outer surface of the cutting end, and a counterbore offset a distance from a distal end of the cutting end; and a locking end provided at an opposing second end of the cannulated shaft from the cutting end.

In some implementations, the kit may further include a rescue dowel including: a tubular body defining a central lumen extending therethrough, the tubular body having a proximal end defining a proximal end opening extending to the central lumen and a distal end defining a distal end opening extending to the central lumen.

In some implementations, the kit may further include a guide pin sized and configured to extend from and frictionally engage with a phalange on a distal side of a patient's joint through at least a portion of a phalange on a proximal side of the patient's joint, wherein the guide pin is further sized and configured to be received within the central lumen of the peg reamer and the central lumen of the hole reamer.

In some implementations, the techniques described herein relate to a method for straightening an interphalangeal joint, including: accessing an interphalangeal joint; separating first and second bones on opposite sides of the interphalangeal joint; removing material from one of the first or second bone so as to form a peg and removing material from the other bone so as to form an opposing hole, wherein the peg is sized and configured to be received by and frictionally engage with the hole; positioning a counter-rotation ring around the peg, wherein the counter-rotation ring includes an annular body having a first surface and an opposing second surface, wherein a plurality of axial spikes extend from at least one of the first and second surfaces; inserting at least one axial spike into the first or second bone from which the peg is formed such that the first surface of the annular body abuts a surface that surrounds the peg; placing the first and second bones in an assembled relationship such that the hole receives the peg; inserting at least one axial spike into the first or second bone from which the hole is formed such that the second surface of the annular body abuts a surface that surrounds the hole; wherein the counter-rotation ring rotationally fixes the first bone and second bone, thereby facilitating fusion of the assembled first and second bones.

In some implementations, the method may further include defining channels through at least portions of the lengths of both the first and second bones, wherein the channels are sized and configured to receive a guide pin therethrough.

In some implementations, the method may further include introducing the guide pin into the channel of the first bone.

In some implementations, the method may further include introducing the guide pin into the channel of the second bone.

In some implementations, the step of removing material from one of the first or second bone so as to form a peg may further include engaging the guide pin with a central lumen of a peg reamer.

In some implementations, the step of removing material from the other of the first or second bone so as to form an opposing hole may further include engaging the guide pin with a central lumen of a hole reamer.

In some implementations, the techniques described herein relate to a method for straightening an interphalangeal joint, including: accessing an interphalangeal joint; separating bones on opposite sides of the interphalangeal joint, wherein a first bone is proximal relative to the joint and a second bone is distal relative to the joint; removing material from one of the first or second bone so as to form a hole and removing material from the other bone so as to form an opposing hole; inserting a rescue dowel into the hole formed in one of the first or second bone, the rescue dowel including a tubular body sized and configured to be received by and frictionally engage with said hole, the tubular body defining a lumen therethrough; positioning a counter-rotation ring around the rescue dowel, wherein the counter-rotation ring includes an annular body having a first surface and an opposing second surface, wherein a plurality of axial spikes extends from at least one of the first and second surfaces; inserting at least one axial spike into the first or second bone from which the rescue dowel is first inserted such that the first surface of the annular body abuts a surface that surrounds the rescue dowel; placing the first and second bones in an assembled relationship such that the hole of the other of the first or second bone receives the rescue dowel; inserting at least one axial spike into said other of the first or second bone such that the second surface of the annular body abuts a surface that surrounds the hole of said other of the first or second bone; wherein the counter-rotation ring rotationally fixes the first bone and second bone, thereby facilitating fusion of the assembled first and second bones.

In some implementations, the method may further include defining channels through at least portions of the lengths of both the first and second bones, wherein the channels are sized and configured to receive a guide pin therethrough.

In some implementations, the method may further include introducing the guide pin into the channel of the first bone.

In some implementations, the method may further include introducing the guide pin into the channel of the second bone.

In some implementations, the step of removing material from one of the first or second bone so as to form a hole may further include engaging the guide pin with a central lumen of a hole reamer.

In some implementations, the step of placing the first and second bones in an assembled relationship such that the hole of the other of the first or second bone receives the rescue dowel may further comprise engaging the guide pin with the lumen of the rescue dowel.

In some implementations, the step of removing material from the other of the first or second bone so as to form an opposing hole may further include engaging the guide pin with a central lumen of a hole reamer.

This configuration can be provided with any one or more of the features described elsewhere herein. This somewhat basic configuration can be provided with any one or more of the features shown in the figures and/or described in conjunction with the figures, either in addition to or alternatively to the features of the examples described hereafter.

### BRIEF DESCRIPTION OF DRAWINGS

FIGS. 1A-1D are various views of an exemplary counter-rotation ring. FIG. 1A is a perspective view of the counter-rotation ring. FIG. 1B is a side view of the counter-rotation ring shown in FIG. 1A. FIG. 1C is a top view of the counter-rotation ring shown in FIG. 1A. FIG. 1D is a cross-sectional view of the counter-rotation ring shown in FIG. 1C taken along section line A-A.

FIGS. 2A-2D are various views of another exemplary counter-rotation ring. FIG. 2A is a perspective view of the counter-rotation ring. FIG. 2B is a side view of the counter-rotation ring shown in FIG. 2A. FIG. 2C is a top view of the counter-rotation ring shown in FIG. 2A.

FIG. 2D is a cross-sectional view of the counter-rotation ring shown in FIG. 2C taken along line A-A.

FIG. 3A is a perspective view of another exemplary counter-rotation ring. FIG. 3B is a cross-sectional side view of the counter-rotation ring shown in FIG. 3A.

FIG. 4A is a perspective view of another exemplary counter-rotation ring. FIG. 4B is a cross-sectional side view of the counter-rotation ring shown in FIG. 4A.

FIG. 5A is a perspective view of another exemplary counter-rotation ring. FIG. 5B is a cross-sectional side view of the counter-rotation ring shown in FIG. 5A.

FIG. 6A is a perspective view of another exemplary counter-rotation ring. FIG. 6B is a cross-sectional side view of the counter-rotation ring shown in FIG. 6A.

FIG. 7A is a perspective view of another exemplary counter-rotation ring. FIG. 7B is a cross-sectional side view of the counter-rotation ring shown in FIG. 7A.

FIG. 8A is a perspective view of another exemplary counter-rotation ring. FIG. 8B is a cross-sectional side view of the counter-rotation ring shown in FIG. 8A.

FIGS. 9A-9E are perspective views of additional exemplary counter-rotation rings.

FIG. 10 is a perspective view of another exemplary counter-rotation ring.

FIGS. 11A-11B are perspective views of additional exemplary counter-rotation rings.

FIG. 12 is a perspective view of another exemplary counter-rotation ring.

FIG. 13 is a perspective view of another exemplary counter-rotation ring.

FIG. 14 is a perspective view of another exemplary counter-rotation ring.

FIG. 15 is a perspective view of another exemplary counter-rotation ring.

FIG. 16 is a perspective view of another exemplary counter-rotation ring.

FIG. 17 shows side views of another exemplary counter-rotation ring.

FIGS. 18A-18E are various views of an exemplary peg reamer. FIG. 18A is a side view of the exemplary peg reamer. FIG. 18B is a cross-sectional side view of the exemplary peg reamer shown in FIG. 18A taken along section line A-A. FIG. 18C is a front view of the exemplary peg reamer shown in FIG. 18A. FIG. 18D is a cross-sectional side view of the exemplary peg reamer shown in FIG. 18C taken along section line B-B. FIG. 18E is a perspective view of the exemplary peg reamer shown in FIG. 18A.

FIGS. 19A-19B are side and perspective views of additional exemplary peg reamers.

FIGS. 20A-20B show an exemplary peg reamer shaping a bone.

FIGS. 21A-21G are various views of an exemplary hole reamer.

FIG. 22 is a side view of another exemplary hole reamer.

FIG. 23 is a perspective view of another exemplary hole reamer.

FIGS. 24A-24B show an exemplary hole reamer shaping a bone.

FIGS. 25A-25D show an exemplary interphalangeal joint fusion system including a counter-rotation ring and a guide pin positioned relative to bones of a patient's foot.

FIG. 26 is an exploded side view of the exemplary interphalangeal joint fusion system shown in FIGS. 25A-25D.

FIG. 27 shows an exemplary gripping tool gripping an exemplary counter-rotation ring.

FIG. 28 is a perspective view of an exemplary recovery device.

FIG. 29 is a perspective view of another exemplary recovery device.

FIG. 30 is a perspective view of another exemplary recovery device.

FIG. 31A is a perspective view of another exemplary recovery device. FIG. 31B is a side view of the recovery device shown in FIG. 31A.

FIG. 32A is a perspective view of another exemplary recovery device. FIG. 32B is a side view of the recovery device shown in FIG. 32A.

FIG. 33 is a perspective view of another exemplary recovery device.

FIG. 34A is a perspective view of another exemplary recovery device. FIG. 34B is a cross-sectional side view of the recovery device shown in FIG. 34A.

FIGS. 35A-35C show an exemplary interphalangeal joint fusion system including a counter-rotation ring, a recovery device, and a guide pin positioned relative to bones of a patient's foot.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

As previously described, the present disclosure relates to techniques and devices used in interphalangeal joint fusion medical procedures. The interphalangeal joint fusion systems described herein can be used to prepare one or more bones at a procedure site to facilitate arthrodesis (hereinafter referred to as “bone fusion”). Furthermore, the interphalangeal joint fusion systems described herein can include one or more medical devices that are implanted at the procedure site that facilitate bone fusion. As provided herein, the interphalangeal joint fusion system can be used to stabilize bones on opposing sides of an interphalangeal joint to aid in the fixation of fractures, fusions, and osteotomies of phalanges.

### Counter-Rotation Ring

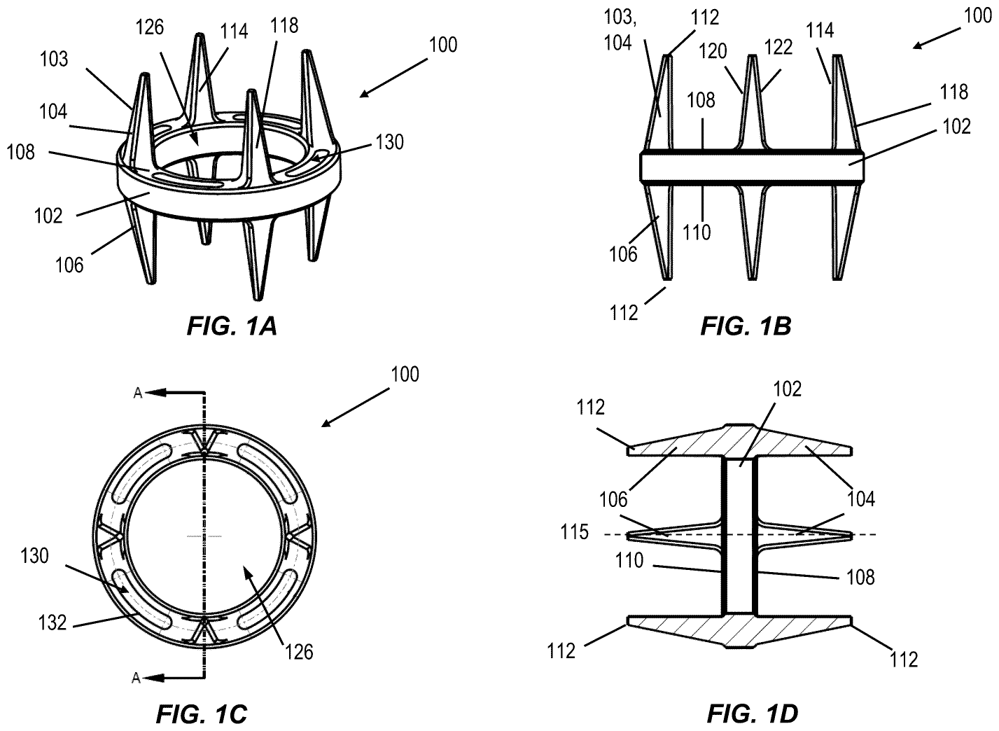

In one implementation, the interphalangeal joint fusion system includes a counter-rotation ring 100 that is sized and configured to resist relative rotational movement of two bones to be fused. FIG. 1A illustrates an example counter-rotation ring 100 that includes annular body 102 having a proximal surface 108 and an opposing distal surface 110 and defining a central lumen 126. A central axis 115 is defined through the central lumen 126 of the counter-rotation ring 100 (as shown in FIG. 1C).

As shown in FIG. 1A, the annular body 102 includes an axial spike 103 that extends axially from at least one of the proximal surface 108 or distal surface 110 of the annular body 102. As further described herein, the axial spike 103 can be in the form of a proximal axial spike 104 that extends proximally from the proximal surface 108 and/or a distal axial spike 106 that extends distally from the distal surface 110. As used herein, proximal axial spikes 104 and distal axial spikes 106 may be collectively referred to as axial spikes 103 when describing attributes that are common to both sets of axial spikes.

As shown in FIGS. 1A-1D, the counter-rotation ring 100 can include a plurality of proximal axial spikes 104 and a plurality of distal axial spikes 106. Specifically, the illustrated example includes four proximal axial spikes 104 and four distal axial spikes 106. As further described herein, the axial spikes 103 are configured to anchor the counter-rotation ring 100 into bone, remain therein, and resist relative rotational movement of two bones to be fused. Thus, in some implementations, other numbers of axial spikes 104 and/or distal axial spikes 106 may be used, so long as the counter-rotation ring 100 is able to penetrate into the bone of a patient and remain anchored therein.

In the illustrated implementation, the plurality of proximal spikes 104 are arranged circumferentially about the proximal surface 108 of the annular body 102. Similarly, the plurality of distal spikes 106 is arranged circumferentially about the distal surface 110 of the annular body 102. Furthermore, the plurality of proximal spikes 104 and the plurality of distal spikes 106 are evenly spaced about the corresponding proximal surface 108 and distal surface 110. As shown in FIG. 1B, the circumferential location of each one of the plurality of the proximal spikes 104 on the proximal surface 108 can correspond with the circumferential location of a corresponding one of the plurality of distal spikes 106 on the distal surface 110.

As shown in FIGS. 1A and 1B, each axial spike 103 is defined between an inner surface 114 and an opposite outer surface 118, where the inner surface 114 is radially inward of the outer surface 118. In some implementations, at least one of the proximal spike 104 or the distal spike 106 can have an inner surface 114 that extends generally parallel with the central axis 115 of the annular body 102. Furthermore, in some implementations, at least one of the proximal spike 104 or the distal spike 106 can have an outer surface 118 that has a decreasing taper from a corresponding proximal surface 108 or distal surface 110 of the annular body toward the spike tip 112 of the proximal spike 104 or distal spike 106.

In the illustrated example, the outer surface 118 is angled radially inward toward the central axis 115 of the annular body 102. In other words, the outer surface 118 is angled radially inward toward an axial spike tip 112 opposite the annular body 102, while the inner surface 114 extends perpendicular to the annular body 102. Specifically, the inner surfaces 114 of the proximal axial spikes 104 extend perpendicular to the proximal surface 108 of the annular body 102 and the inner surfaces 114 of the distal axial spikes 106 extend perpendicular to the distal surface 110 of the annular body 102. Meanwhile, the outer surfaces 118 of the proximal axial spikes 104 and distal axial spikes 106 slope radially inward toward the central axis 115. Furthermore, as shown in FIG. 1B, each axial spike 103 ends in an axial spike tip 112 opposite the annular body 102. As shown in the illustrated implementation, a radial thickness of each axial spike 103 narrows/decreases toward the axial spike tip 112 as the outer surface 118 slopes toward the inner surface 114. In some implementations, the angled inner surface 114 helps to position and/or advance the counter-rotation ring 100 into the bone material.

Each axial spike 103 is further defined by a first side edge 120 and a circumferentially opposite second side edge 122. In some implementations, at least one of the proximal spike 104 or the distal spike 106 can have a decreasing width from the corresponding proximal surface 108 or distal surface 110 of the annular body 102 toward an axial spike tip 112 of the proximal spike 104 or the distal spike 106. As shown in FIG. 1B, the width of the axial spike 103 defined between the first side edge 120 and second side edge 122 and narrows/decreases toward the axial spike tip 112 such that the first side edge 120 and second side edge 122 define an increasing tapered surface from the axial spike tip 112 to the corresponding proximal surface 108 or distal surface 110. In other words, the axial spikes 103 taper in width as they extend axially outward from the annular body 102. In some implementations, the axial spikes 103 can taper at an angle ranging from 6° to an angle of 8°. However, other angles of taper are contemplated, so long as the axial spike 103 facilitates advancement of the counter-rotation ring 100 into the bone material

In the illustrated example, the axial spike tip 112 is blunted. However, this disclosure contemplates many possible shapes to the spike tip 112. For example, the spike tip 112 can extend to a fine or sharp point. As further described herein, the axial spikes 103 are sized and configured to anchor the counter-rotation ring 100 into bone, remain therein, and resist relative rotational movement of two bones to be fused. Accordingly, the axial spikes 103 spike tips 112 can have any number of shapes that facilitate penetration into the bone of a patient and remain anchored therein.

The counter-rotation ring 100 can be dimensioned to account for variations in anatomy and patient size. Accordingly, various implementations of the counter-rotation ring 100 can have a range of dimensions, such as for accommodating both adult and pediatric patients. In some implementations, the counter-rotation ring 100 can have a length ranging from 6.0 mm to 7.0 mm from the axial spike tips 112 of the proximal axial spikes 104 to the axial spike tips 112 of the distal axial spikes 106. For example, the length of the counter-rotation ring 10 can be 6.5 mm.

Furthermore, the outer diameter of the annular body 102 can also range from 6.0 mm to 7.0 mm. For example, the outer diameter of the annular body 102 can be 6.5 mm. Additionally, in some implementations, the annular body 102 can have a thickness defined between the proximal surface 108 and the distal surface 110, where the thickness ranges from 0.5 mm to 1.5 mm. For example, the thickness can range from 0.8 mm to 1.2 mm. In some implementations, the thickness of the annular body 102 can be about 1.0 mm.

As described further herein, the central lumen 126 of the annular body 102 is sized and configured to receive a peg formed from patient bone or an artificial material such as the rescue dowel 500 further described below. Accordingly, the diameter of the central lumen 126 corresponds to the diameter of the peg. In various examples, the diameter of the central lumen 126 can range from 4.0 mm to 5.0 mm. In some implementations, the inner diameter of central lumen 126 can be about 4.0 mm.

In some implementations, the annular body 102 can have a generally circular-shaped outer surface 125 and a generally circular-shaped inner surface radially inward relative to the outer surface 125. In this way, the annular body 102 can have a generally rectangular-shaped cross-section. In some implementations, the annular body 102 can have a rectangular cross-section ranging from about 3.60 mm\hat{ }2 to 3.70 mm\hat{ }2. In some examples, the rectangular cross-section of the annular body 102 can be about 3.63 mm\hat{ }2. In further examples, the rectangular cross-section of the annular body 102 can be about 3.65 mm\hat{ }2.

As shown in FIG. 1C, the annular body 102 defines a plurality of circumferential slots 130 arranged circumferentially about the annular body 102 and extending at least partially through the annular body 102. In the illustrated example, the circumferential slots 130 extend between the proximal surface 108 and the distal surface 110 of the annular body 102 and are spaced evenly about a circumference of the annular body 102. Furthermore, in the illustrated implementation, each circumferential slot 130 includes an elongated body portion 132 that has a curvature corresponding to a curvature of the annular body 102. In some implementations, the circumferential slots 130 facilitate bone ingrowth into the counter-rotation ring 100, thereby improving stability of fused joint.

In some implementations, the counter-rotation ring 100 can include features that further facilitate anchoring the counter-rotation ring 100 within the bone of the patient. For example, in various implementations, at least one of the proximal spike 104 or distal spike includes a barb.

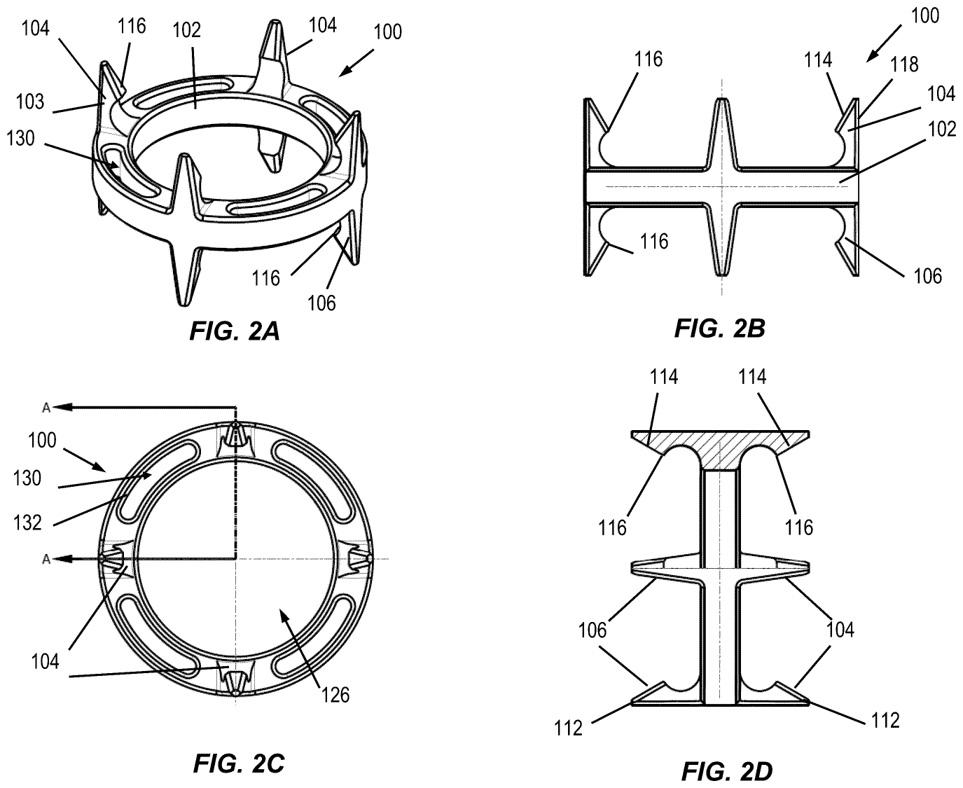

FIGS. 2A-13 show various examples of the counter-rotation ring 100. The counter-rotation ring 100 in FIGS. 2A-13 is similar to the counter-rotation ring 100 described above in reference to FIGS. 1A-1D. Thus, similar reference numbers as those used for the implementation shown in FIGS. 1A-1D are used to reference similar features of the implementation shown in FIGS. 2A-13. Furthermore, any features in any other implementations disclosed herein can be included in the implementation disclosed in FIGS. 1A-13.

FIG. 2A-2D show an exemplary counter-rotation ring 100 that includes barbs 116 extending radially inward from the inner surfaces 114 of the proximal axial spikes 104 and distal axial spikes 106. Additionally, in this implementation, the outer surfaces 118 are generally parallel with the central 115 axis of the counter-rotation ring 100. In the illustrated implementation, the barbs 116 are angled radially inward toward the central axis 115 of the counter-rotation ring 100 at an angle of 30°. Furthermore, while a portion of the inner surface 114 that extends between the axial spike tip 112 and the radially inward barb 116 is planar, a portion of the inner surface 114 that extends between the radially inward barb 116 and the respective proximal surface 108 and distal surface 110 is curved. In various implementations, the radially inward barb 116 helps prevent pullout/withdraw of the counter-rotation ring 100 from the bone material, thereby improving anchoring of the counter-rotation ring 100 within the bone. Accordingly, the barbs 116 can be configured at any number of angles so long as they help resist withdraw of the counter-rotation ring 100.

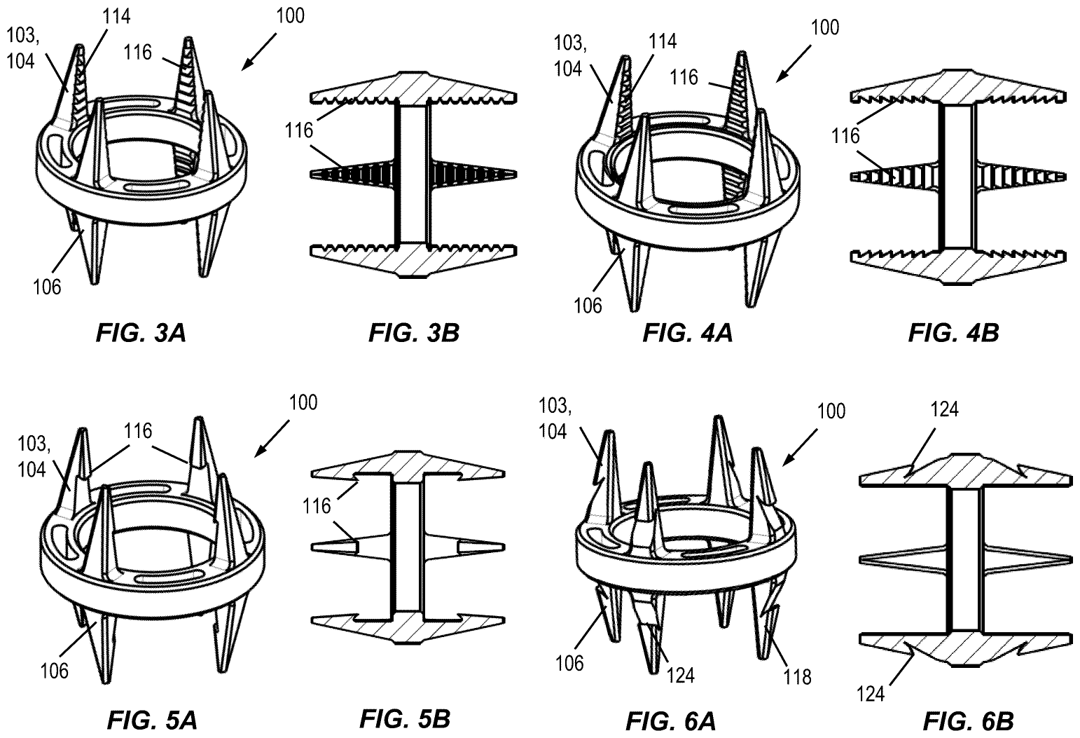

FIGS. 3A-3B show an exemplary counter-rotation ring 100 similar to the counter-rotation ring described above in reference to FIGS. 1A-1D. However, this exemplary counter-rotation ring 100 includes a plurality of radially inwardly extending barbs 116 to improve anchoring and inhibit pullout of the counter-rotation ring 100 from the bone. Specifically, FIGS. 3A-3B show a plurality of relatively small barbs 116 extending radially inward in a direction normal to the inner surface 114 of the axial spike 103.

FIGS. 4A-4B show an exemplary counter-rotation ring 100 similar to the counter-rotation ring described above in reference to FIGS. 3A-3D. However, the plurality of radially inwardly extending barbs 116 are angled toward the annular body 102. In other words, the radially inwardly extending barbs 116 of the proximal axial spikes 104 are angled toward the proximal surface 108 of the annular body 102, and the radially inwardly extending barbs 116 of the distal axial spikes 106 are angled toward the distal surface 110 of the annular body 102.

FIGS. 5A-5B show an exemplary counter-rotation ring 100 similar to the counter-rotation ring described above in reference to FIGS. 1A-1D. However, this exemplary counter-rotation ring 100 includes a plurality of radially inwardly extending barbs 116 similar to the radially inward barbs of FIGS. 2A-2D. However, unlike the inwardly extending barbs 116 of FIGS. 2A-2D, in this implementation, the portion of the inner surface 114 that extends between the radially inward barb 116 and the respective proximal surface 108 and distal surface 110 is generally parallel with the central axis 115 of the counter-rotation ring 100.

FIGS. 6A-6B show an exemplary counter-rotation ring 100 similar to the counter-rotation ring described above in reference to FIGS. 1A-1D. However, this exemplary counter-rotation ring 100 includes a plurality of barbs 124 extending radially outward from the outer surface 118 away from the central axis 115 of the counter-rotation ring 100. As shown, the barbs 124 slope axially inward toward the respective proximal surface 108 and distal surface 110 of the counter-rotation ring 100.

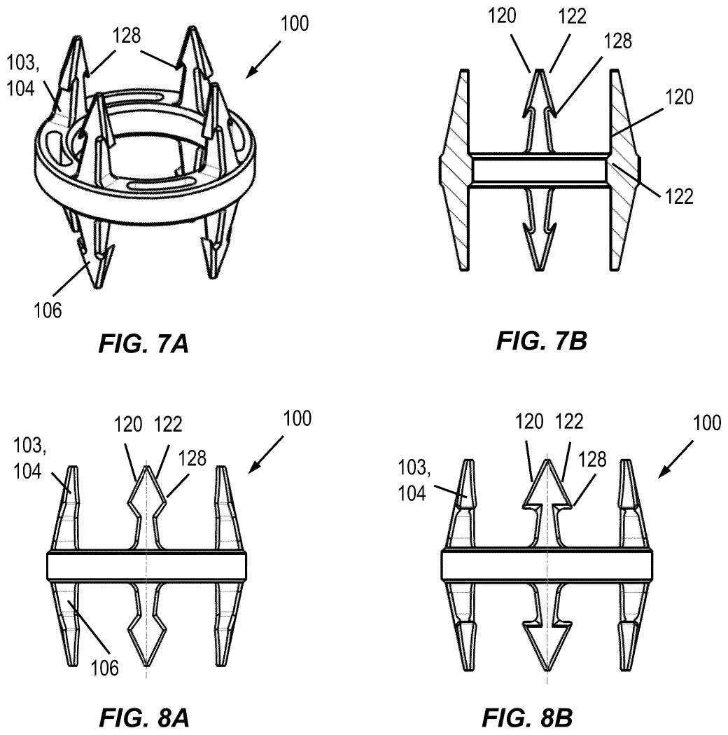

FIGS. 7A-7B show an exemplary counter-rotation ring 100 similar to the counter-rotation ring described above in reference to FIGS. 2A-2D. However, the exemplary counter-rotation ring 100 illustrated in FIGS. 7A-7D includes barbs 128 extending circumferentially outward from the first and second side edges 120, 122 of the proximal spikes 104 and distal spikes 106. In the illustrated implementation, the barbs 128 slope axially inward toward the respective proximal surface 108 and distal surface 110 but the inner surface 114 remains generally parallel with the central axis 115 of the counter-rotation ring 100 (i.e., perpendicular to the respective proximal surface 108 and distal surface 110). In other words, the circumferentially extending barb 128 has an arrow-shaped configuration where the bases of the arrow are angled with respect to the central axis 115 of the counter-rotation ring 100.

FIG. 8A shows an exemplary counter-rotation ring 100 similar to the counter-rotation ring described above in reference to FIGS. 7A-7D. However, a portion of the first and second side edges 120, 122 that extends between the axial spike tip 112 and the circumferentially extending barb 128 is angled axially inward toward the respective proximal surface 108 and distal surface 110. As illustrated in FIG. 8A, a portion of the first and second side edges 120, 122 that extends between the respective proximal surface 108 and distal surface 110 and the circumferentially extending barb 128 is angled axially outward toward the axial spike tip 112. In this way, the circumferentially extending barb 128 has a diamond-shaped configuration.

FIG. 8B shows an exemplary counter-rotation ring 100 similar to the counter-rotation ring described above in reference to FIG. 8A. However, the portion of the first and second side edges 120, 122 that extends between the respective proximal surface 108 and distal surface 110 and the circumferentially extending barb 128 is angled generally parallel to the respective proximal surface 108 and distal surface 110. In other words, the circumferentially extending barb 128 has an arrow-shaped configuration where the bases of the arrow are generally perpendicular to the central axis 115 of the counter-rotation ring 100.

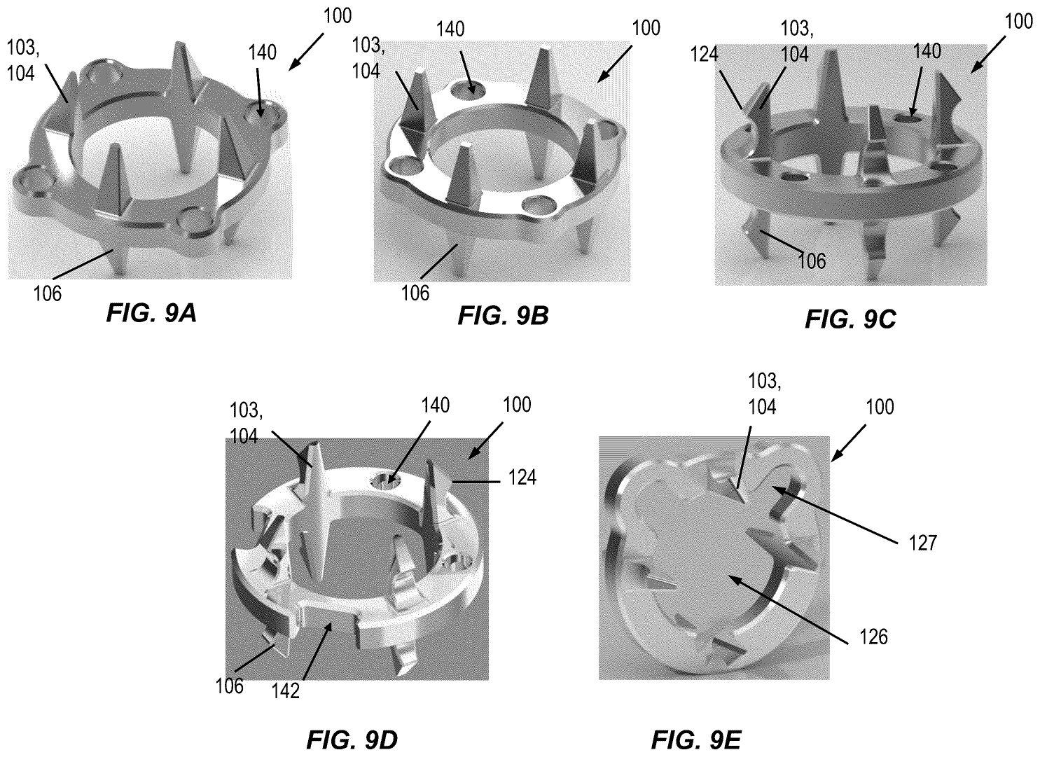

FIG. 9A shows an exemplary counter-rotation ring 100 similar to the counter-rotation ring described above in reference to FIGS. 1A-1D. However, the annular body 102 further defines a suture opening 140 extending through the annular body 102. Specifically, the illustrated implementation shows four suture openings 140 arranged circumferentially about the annular body 102. In some implementations, the suture openings 140 are spaced equidistant from the axial spikes 103. As illustrated in FIG. 9A, the suture openings 140 can be included on a portion of the annular body 102 that extends partially radially outward from the main body portion annular body 102. As such, the portion of annular body 102 including the suture openings 140 has a larger diameter than the main body portion of the annular body 102. In use, sutures can be passed through the suture openings 140 to help secure the counter-rotation ring 100 to the patient's bone.

FIG. 9B shows an exemplary counter-rotation ring 100 similar to the counter-rotation ring described above in reference to 9A. However, both the inner surface 114 and outer surface 118 of each of the proximal axial spikes 104 and distal axial spikes 106 angled toward the axial spike tip 112.

FIG. 9C shows an exemplary counter-rotation ring 100 similar to the counter-rotation ring described above in reference to FIGS. 2A-2D. However, the exemplary counter-rotation ring 100 illustrated in FIG. 9C includes barbs 124, which instead extend radially outward from the outer surfaces 118. Unlike the implementation shown in FIGS. 2A-2D, the inner surfaces 114 of the axial spikes 103 in this implementation are generally parallel with the central axis 115 of the counter-rotation ring 100. Furthermore, as shown in the illustrated implementation, the annular body 102 defines suture openings 140 arranged circumferentially about the annular body 102 and extending therethrough. Specifically, the suture openings 140 are defined between the outer and inner surfaces of the annular body 102, such that the outer surface 125 of the annular body 102 has a constant diameter.

FIG. 9D shows an exemplary counter-rotation ring 100 similar to the counter-rotation ring described above in reference to FIG. 9C. However, the exemplary counter-rotation ring 100 illustrated in FIG. 9D includes a staple cutout 142 extending radially inward from the outer surface 125 of the annular body 102 toward the central axis 115 of the annular body 102. Specifically, the illustrated implementation includes two staple cutouts 142. In the implementation shown in FIG. 9D, the staple cutouts 142 are positioned asymmetrically around the annular body 102. For example, the staple cutouts 142 can be positioned within the same 180° segment of the annular body 102. These staple cutouts 142 facilitate anchoring the counter-rotation ring 100 to the patient's bone using a surgical staple.

FIG. 9E shows an exemplary counter-rotation ring 100 similar to the counter-rotation ring described above in reference to FIG. 9C. However, the inner surface of the annular body 102 defines radial projections 127 extending radially away from the central axis 115 of the annular body 102. Specifically, as shown in the illustrated implementation, the radial projections 127 are continuous with the central lumen 126. The radial projections 127 may be referred to herein as “bear ears.” In the implementation shown in FIG. 9E, the radial projections 127 are positioned asymmetrically around the annular body 102. For example, as provided in FIG. 9D, the radial projections 127 are positioned within the same 180° segment of the annular body 102. Similar to the staple cutouts 142, the radial projections 127 can further facilitate suturing the counter-rotation ring 100 to the patient's bone.

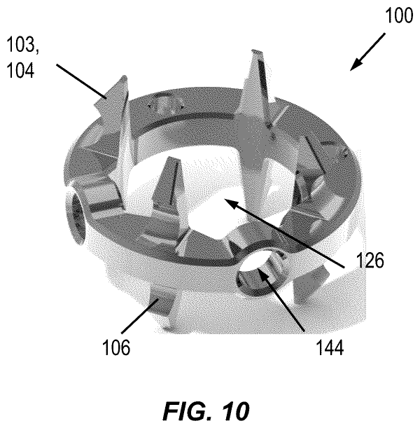

FIG. 10 shows an exemplary counter-rotation ring 100 similar to the counter-rotation ring described above in reference to FIG. 9C. However, the exemplary counter-rotation ring 100 illustrated in FIG. 10 includes a side opening 144 extending radially inward from the outer surface 125 of the annular body 102 toward the central axis 115 of the annular body 102. In various implementations, the side opening 144 allows for advancement of a fixation pin or screw through the annular body 102 of the counter-rotation ring 100 and into a peg formed from patient bone or a rescue dowel 500 (described below). Thus, the counter-rotation ring 100 can be further rotationally and axially fixed to the peg of bone or rescue dowel 500.

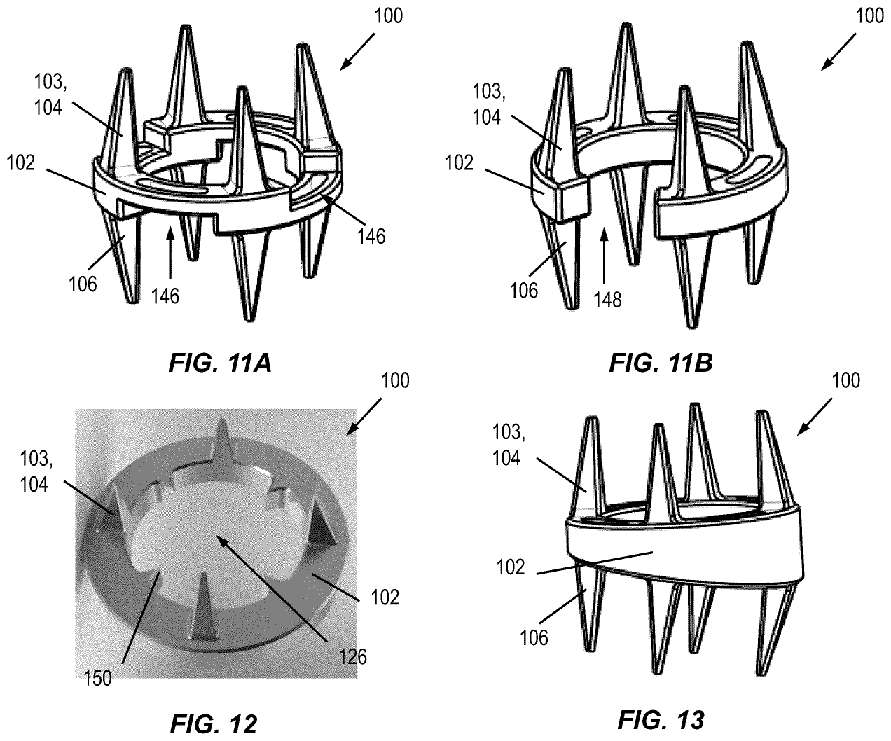

FIG. 11A shows an exemplary counter-rotation ring 100 similar to the counter-rotation ring described above in reference to FIGS. 1A-1D. However, the exemplary counter-rotation ring 100 illustrated in FIG. 11A includes negative spaces 146 extending axially inward from respective proximal and distal surfaces 108, 110 of the annular ring 102. In other words, the proximal surface 108 defines negative spaces 146 extending axially toward the distal surface 110, and the distal surface 110 defines negative spaces 146 extending axially toward the distal surface 108.

FIG. 11B shows an exemplary counter-rotation ring 100 similar to the counter-rotation ring described above in reference to FIGS. 1A-1D. However, the exemplary counter-rotation ring 100 illustrated in FIG. 11B includes a gap 148 defined within the annular ring 102 such that the annular ring 102 extends circumferentially between a first end and a second end. The gap 148 provides a space into which bone on either side of the interphalangeal can grow, thereby further stabilizing the counter-rotation ring 100 relative to the bones.

FIG. 12 shows an exemplary counter-rotation ring 100 similar to the counter-rotation ring described above in reference to FIGS. 1A-1D. However, the exemplary counter-rotation ring 100 illustrated in FIG. 12 includes spikes 150 extending radially inward from the inner surface of the annular ring 102. These radially inward spikes 150 can help the counter-rotation ring 100 grip the peg of bone, thereby reducing the risk that the counter-rotation ring 100 axially dislodges from the peg of bone. The radially inward spikes 150 can also help prevent the counter-rotation ring 100 from rotating relative to the peg of bone.

FIG. 13 shows an exemplary counter-rotation ring 100 similar to the counter-rotation ring described above in reference to FIGS. 1A-1D. However, the thickness of the annular ring varies such that the proximal surface 108 and distal surface 110 are angled relative to each other. Accordingly, the distance between axial spike tips 112 of some pairs of proximal axial spikes 104 and corresponding distal axial spikes 106 can be greater than the distance between axial spike tips 112 of other pairs of proximal axial spikes 104 and corresponding distal axial spikes 106. This oblique structure can facilitate coupling of bones on either side of a joint where a coupling surface of one bone is not complementary to a coupling surface of the other bone.

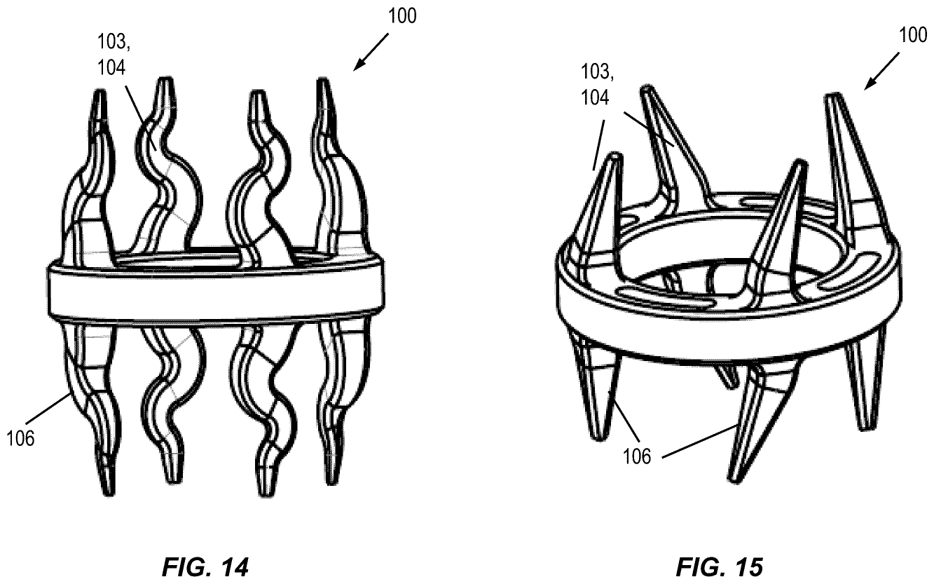

FIG. 14 shows an exemplary counter-rotation ring 100 similar to the counter-rotation ring described above in reference to FIGS. 1A-1D. However, the proximal axial spikes 104 and distal axial spikes 106 extend in a waved or serpentine shape. In other words, the proximal axial spikes 104 and distal axial spikes 106 undulate between the respective proximal surface 108/distal surface 110 and axial spike tip 112.

FIG. 15 also shows an exemplary counter-rotation ring 100 similar to the counter-rotation ring described above in reference to FIGS. 1A-1D. However, in this implementation, at least one of the proximal axial spikes 104 and distal axial spikes 106 are angled circumferentially. That is, the side edges 120, 122 of the proximal axial spikes 104 and distal axial spikes 106 are angled in a circumferential direction. Specifically, in the illustrated implantation, the circumferentially-angled proximal axial spikes 104 are arranged circumferentially at locations corresponding to the locations of the circumferentially-angled distal axial spikes 106. Furthermore, corresponding proximal axial spikes and distal axial spikes 106 extend in circumferential directions that are opposite from each other.

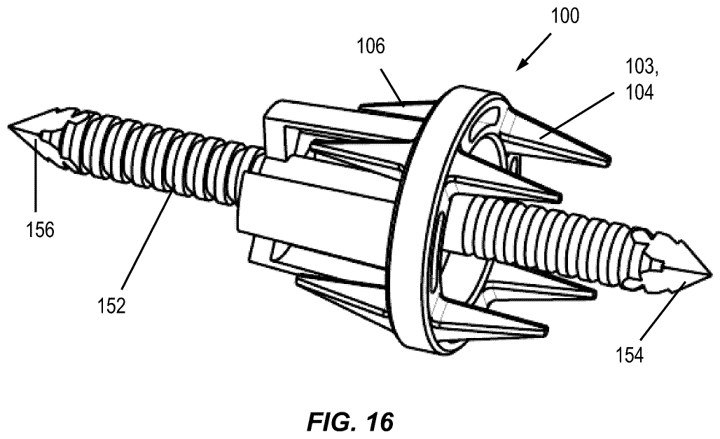

FIG. 16 shows an exemplary counter-rotation ring 100 similar to the counter-rotation ring described above in reference to FIGS. 1A-1D. However, the exemplary counter-rotation ring 100 illustrated in FIG. 16 further includes an axial barb 152 extending through the central lumen 126 of the annular body 102. The axial barb 152 includes a proximal end 154 and a distal end 156. In the illustrated example, both the proximal end 154 and distal end 156 are sharp to facilitate advancement of the axial barb 152 into the bone of a patient. Furthermore, as illustrated in FIG. 16, the axial barb 152 can include ribs to improve retention within the bone.

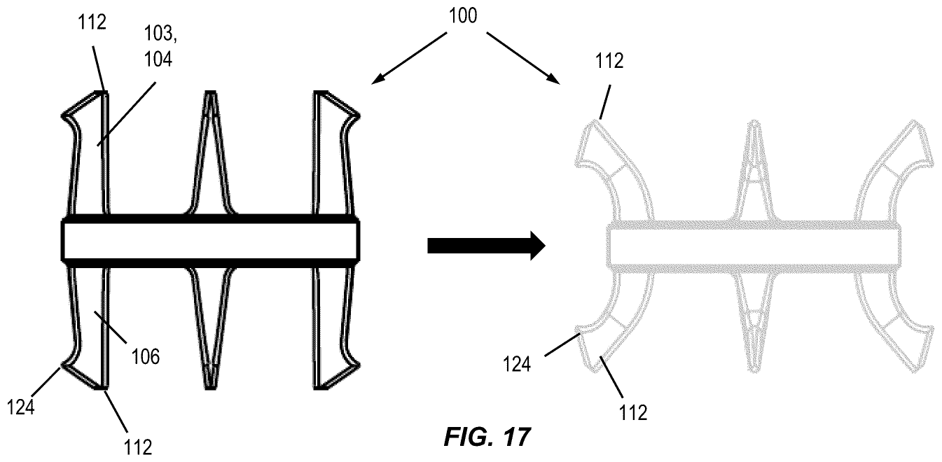

The counter-rotation ring 100 can be formed various biocompatible materials. For example, in some implementations, the counter-rotation ring 100 can be formed from a polymer, including resorbable and non-resorbable polymers as well as co-polymers. In further implementations, the counter-rotation ring 100 can be formed from a metal, such as nitinol, stainless steel, titanium and/or a titanium alloy. For example, FIG. 17 shows an exemplary counter-rotation ring 100 similar to the counter-rotation ring illustrated in FIG. 9C. However, the counter-rotation ring 100 is formed from nitinol. As shown in FIG. 17, the proximal axial spikes 104 and distal axial spikes 106 are heat-set to extend generally parallel with the central axis 115 of the counter-rotation ring 100 in a first configuration. Then, upon a predetermined change in temperature, the counter-rotation ring 100 moves into a second configuration, in which the proximal axial spikes 104 and distal axial spikes 106 bend radially outward. For example, in some implementations, the counter-rotation ring 100 transitions to/toward the second configuration at or around human body temperature. In some examples, including the illustrated implementation, radially outward barbs 124 on the proximal axial spikes 104 and distal axial spikes 106 move toward the respective proximal surface 108 and distal surface 110 of the annular body 102.

As provided further herein, the counter-rotation ring 100 can be implanted between two bones within an interphalangeal joint, where the proximal axial spikes 104 are advanced into a bone on the proximal side of the interphalangeal joint and the distal axial spikes 106 are advanced into a bone on the distal side of the interphalangeal joint. As noted above, the counter-rotation ring 100 prevents the bones on either side of the interphalangeal joint from rotating relative to each other. Advantageously, this stabilizes the interface between the two bones, thereby facilitating their fusion.

### Peg Reamer

In some implementations, the interphalangeal joint fusion system can further include a peg reamer 200 for shaping a bone of a patient into a peg sized to be received within the central lumen of the annular body 102. As further described below, the surface of the peg is shaped to complement a hole formed in a bone on an opposing side of the interphalangeal joint.

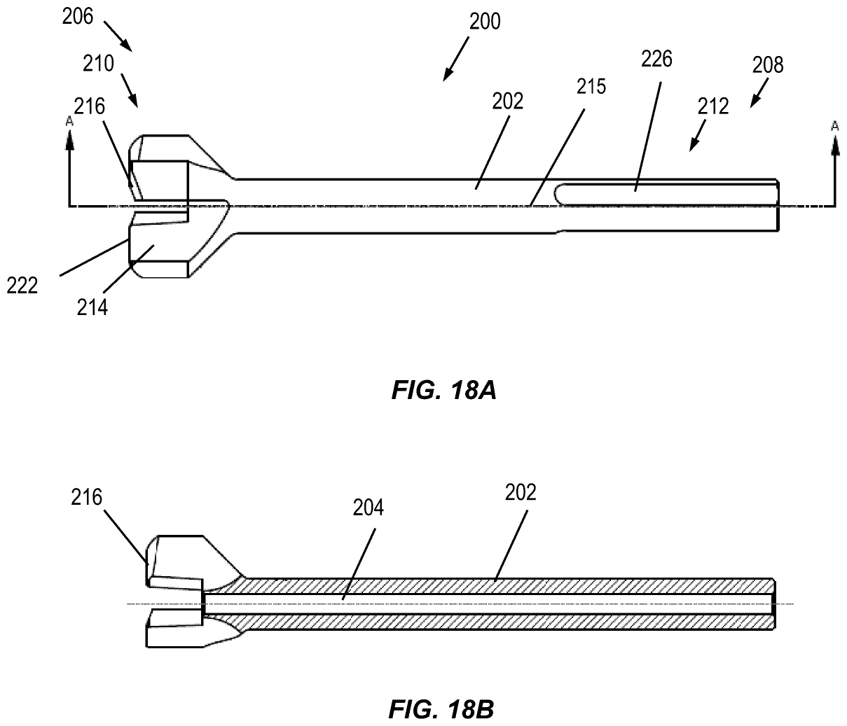

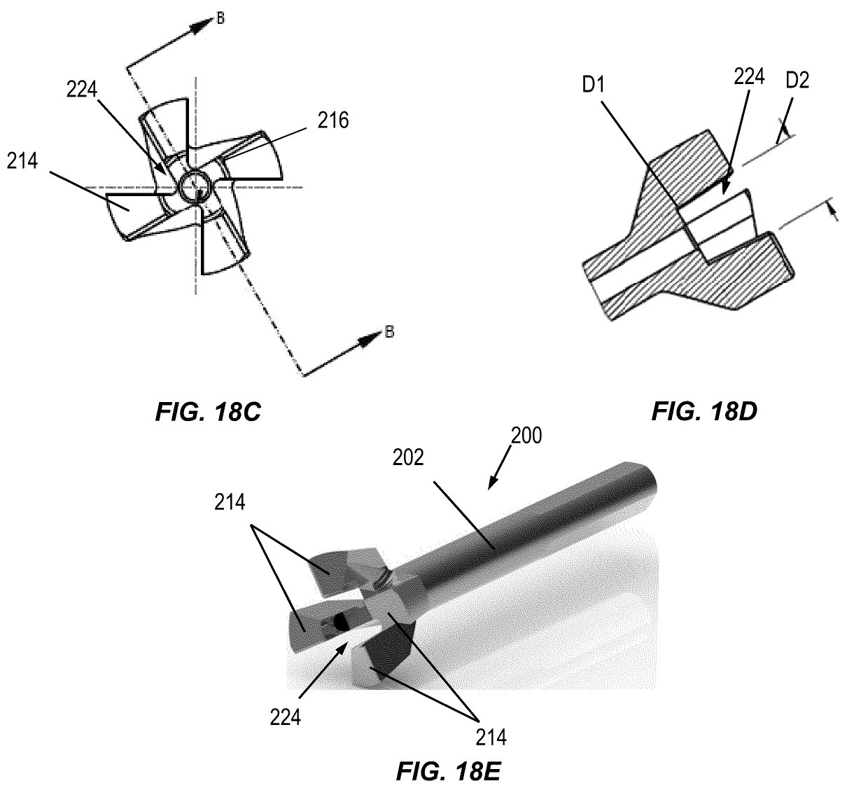

FIGS. 18A-18E show an exemplary peg reamer 200 according to one implementation. As shown in FIG. 18A, the peg reamer 200 includes a cannulated shaft 202 defining a central lumen 204 extending longitudinally therethrough. As shown, the central lumen 204 of the peg reamer 200 cannulated shaft 202 defines a central axis 215 of the peg reamer 200. Furthermore, the cannulated shaft 202 has a first end 206 and a second end 208 that is provided at an opposing end of the cannulated shaft 202 from the first end 206. As shown in FIG. 18B, the central lumen 204 extends an entire length of the cannulated shaft 202 between the first end 206 and the second end 208.

Returning to FIG. 18A, the peg reamer 200 further includes a cutting end 210 provided at the first end 206 of the cannulated shaft 202. The cutting end 206 includes a plurality of cutting arms 214 extending from the cannulated shaft 202.

As shown in FIG. 18C, in the illustrated implementation, the plurality of cutting arms 214 includes four cutting arms 214. However, in further implementations, other numbers of cutting arms 214 may be included, so long as the peg reamer 200 is able to shape an end of a phalange into a peg. As shown, the cutting arms 214 are circumferentially arranged about the cutting end 206 of the peg reamer 200 cannulated shaft 202. Moreover, the plurality of cutting arms 210 are arranged circumferentially around the central axis 215 of the peg reamer 200. Specifically, the plurality of cutting arms 214 are evenly spaced around the central axis 215.

Each cutting arm 214 includes a cutting edge 216 extending along an inner surface of the respective cutting arm 214. In various implementations, including the implementation illustrated in FIGS. 18A-18E (particularly FIG. 18E), each of the cutting edges 216 are spaced apart from the central axis 215 of the cannulated shaft 202. In some implementations, a spacing between each of the cutting edges 216 and the central axis 215 of the peg reamer 200 cannulated shaft 202 can range from 2.25 mm to 2.75 mm. As shown in FIG. 18D, the spacing between each of the cutting edges 216 and the central axis 215 is about 2.56 mm.

In accordance with one aspect of the present disclosure, rotation of the plurality of cutting arms 214 about the central axis 215 of the peg reamer 200 defines a peg cutout region 224 therebetween. As provided herein, the size and shape of the peg cutout region 224 corresponds to the size and shape of the bone material that is retained on the patient's bone forming the peg shape. In this way, rotating the plurality of cutting arms 214 and advancing the cutting end 210 over a target bone shapes the target bone to have a peg shape extending into the interphalangeal joint and matching the peg cutout region 224.

In some implementations, the peg cutout region 224 can have a depth ranging from 4.5 mm to 5.5 mm. In the implementation illustrated in FIG. 18D, the depth of the peg cutout region 224 is about 5 mm. In some implementations, the peg cutout region 224 has a first diameter (D1) adjacent a proximal end of the peg cutout region 224 and a larger second diameter (D2) adjacent a distal end of the peg cutout region 224. In some examples, the second diameter (D2) can range from 5.0 mm to 5.25 mm. In the implementation illustrated in FIG. 18D, the second diameter (D2) is about 5.12 mm. Accordingly, in the illustrated implementation, the cutting edges 216 slope outward relative to the central axis 215 of the peg reamer 200 toward distal ends 222 of the cutting arms 214, wherein the peg cutout region 224 second diameter (D2) is larger than the peg cutout region 224 first diameter (D1) such that the peg cutout region 224 is tapered. This ensures that the resulting peg formed from the patient's bone has a complementary taper.

In some implementations, the central lumen 204 of the peg reamer 200 can be sized and configured to receive a guide pin 400. In some implementations, the central lumen 204 of the peg reamer 200 can have a diameter ranging from 1.5 mm to 2.0 mm. As shown in FIG. 18B, in the illustrated implementation, the diameter of the central lumen 204 is about 1.8 mm.

As further described herein, the peg reamer 200 can be sized and configured to complement a corresponding counter-rotation ring 100, rescue dowel 500, and/or hole reamer 300. For example, the peg reamer 200 can be dimensioned to account for variations in anatomy and patient size. Accordingly, various implementations of the peg reamer 200 can have a range of dimensions, such as for accommodating both adult and pediatric patients.

The peg reamer 200 also includes a locking end 212 provided at the second end 208 of the cannulated shaft 202. The locking end 212 can be configured to engage a drill or other rotating device. The illustrated implementation further includes a flat 226 extending axially along the cannulated shaft 202 at the second end 208. The flat 226 can facilitate stable connection to a drill or other rotating device coupled to the second end 208.

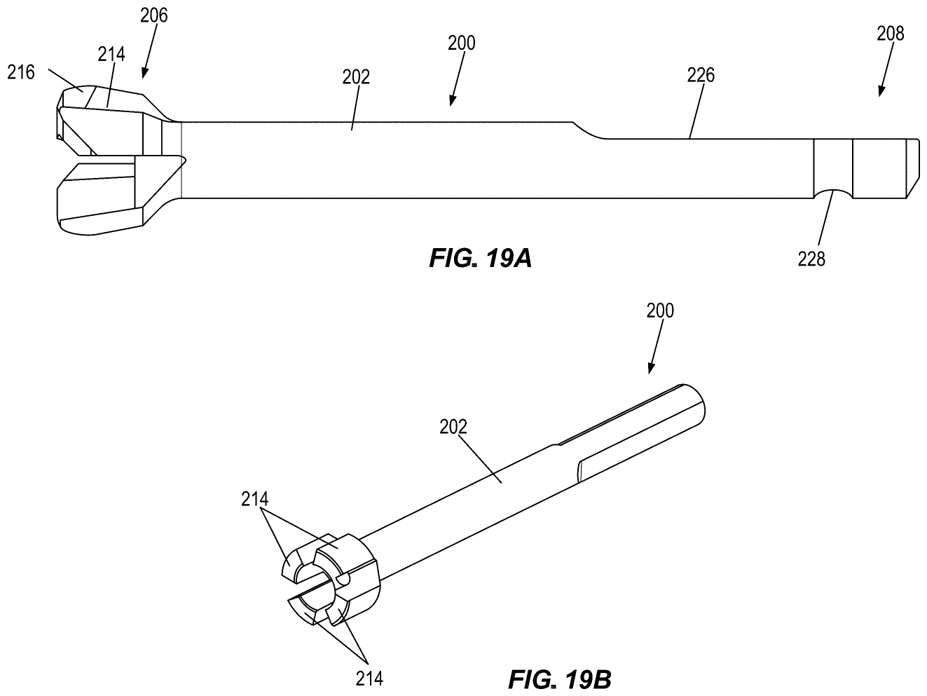

FIGS. 19A-19B show various examples of the peg reamer 200. The peg reamers 200 in FIGS. 19A-19B are similar to the peg reamer 200 described above in reference to FIGS. 18A-18E. Thus, similar reference numbers as those used for the implementation shown in FIGS. 18A-18E are used to reference similar features of the implementation shown in FIGS. 19A-19B. Furthermore, any features in any other implementations disclosed herein can be included in the implementation disclosed in FIGS. 18A-19B.

FIG. 19A shows a side view of an exemplary peg reamer 200 in which the locking end 212 further defines a groove 226 extending circumferentially around the cannulated shaft 202. This groove 226 can further facilitate formation of a stable connection to a drill or other rotating device coupled to the second end 208. For example, the groove 226 can itself provide a means of coupling the peg reamer 200 to a drill or other rotating device.

In some implementations, the peg reamer 200 is formed from stainless steel. However, it is contemplated that the peg reamer 200 can be formed from any material that is capable of removing bone via rotation of the cutting arms 214 and the cutting edges 316 thereon.

FIG. 19B shows a further implementation peg reamer 200 that is similar to the peg reamer described above in reference to FIGS. 18A-18E. However, in this implementation, the cutting arms 214 extend axially away from the cannulated shaft 202 but the cutting edges 216 are generally parallel to the central axis 215 of the peg reamer 200. In other words, the cutting edges 216 are not angled to form a tapered peg cutout region 224. Rather, the peg cutout region 224 is generally cylindrical.

As further described herein, in some implementations, the interphalangeal joint fusion system can include a guide pin 400. The guide pin 400 can be sized and configured to extend from and frictionally engage with a distal bone of a joint (e.g., a distal phalange or second bone) through at least a portion of a proximal bone of the joint (e.g., a proximal phalange or first bone). In some implementations, the guide pin 400 can further be sized and configured to be received within the central lumen 204 of the peg reamer 200 and/or the central lumen 304 of the hole reamer 300. In some implementations, as further described herein, the guide pin 400 can be sized and configured to be received within a central lumen 504 of a rescue dowel 500.

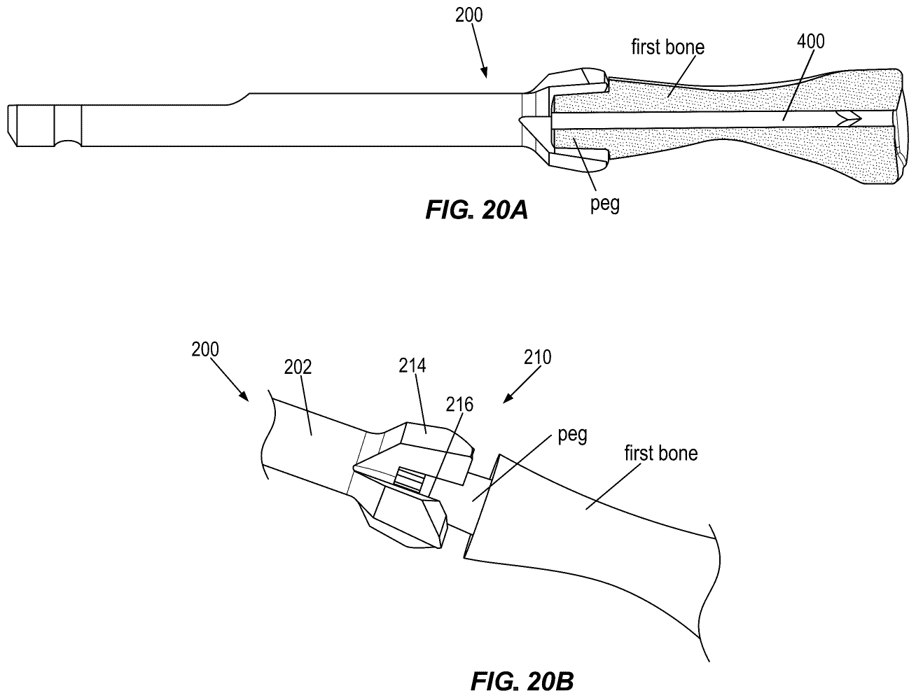

FIGS. 20A-20B show an exemplary peg reamer 200 shaping a bone. As shown, a guide pin 400 is advanced into a first bone along a longitudinal axis of the first bone. Once the guide pin 400 is placed, the peg reamer 200 is aligned with the guide pin 400 such that the central axis 215 of the peg reamer 200 is aligned with the longitudinal axis of a guide pin 400. The peg reamer 200 is then advanced over the guide pin 400 such that the guide pin 400 extends through the central lumen 204 of the peg reamer 200. In this way, the guide pin 400 establishes a path for the peg reamer 200 and defines the location of a subsequent peg formed in the first bone.

As shown in FIG. 20B, rotation of the cutting end 210 of the peg reamer 200 causes the cutting edges 216 to remove bone from a region of the first bone that is outside the peg cutout region 224, thereby forming a peg of bone from the portion of the first bone that remains. In the illustrated implementation, the first bone is a proximal phalanx of a foot. However, in other implementations, the first bone could be another bone on either side of an interphalangeal joint. For example, the peg reamer 200 could be used to shape bone from a middle phalanx of a foot. In further implementations, the peg reamer 200 could be used to shape bone on either side of an interphalangeal joint in a hand.

### Hole Reamer

In some implementations, the interphalangeal joint fusion system can further include a hole reamer 300 for shaping a bone of a patient into define a hole or cavity in the bone. The surface of the hole is shaped to complement the surface of a peg formed in a bone on an opposing side of the interphalangeal joint.

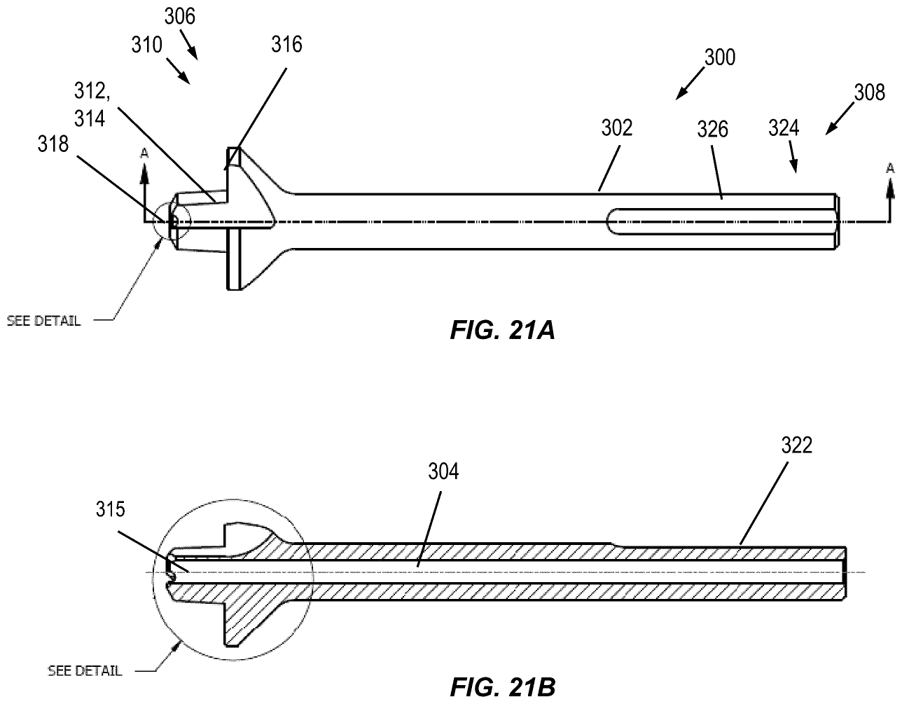

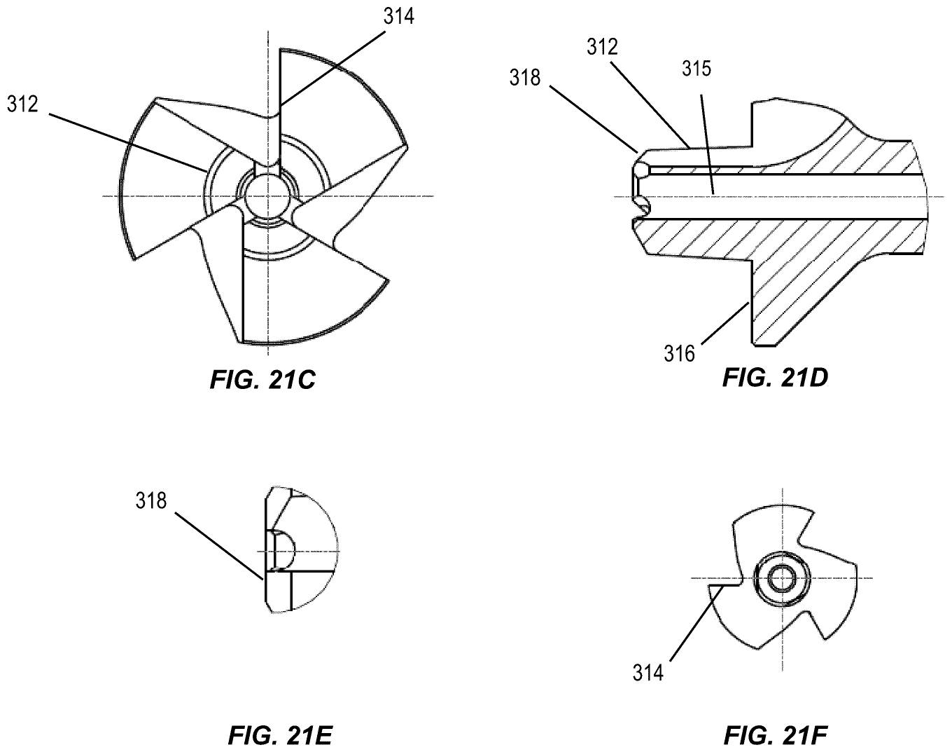

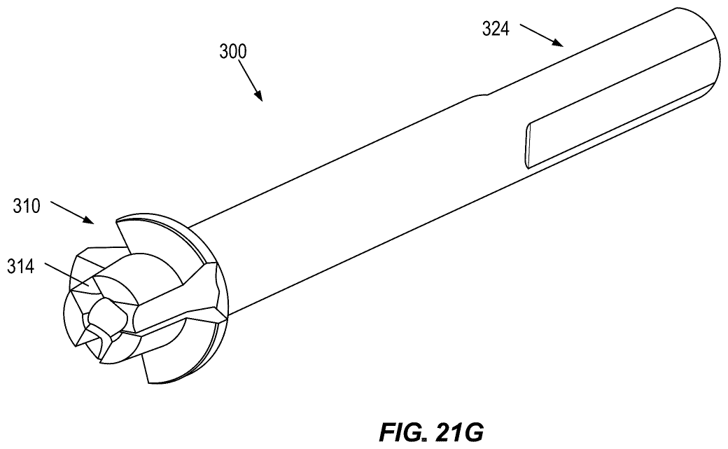

FIGS. 21A-21G show an exemplary hole reamer 300 according to one implementation. As shown in FIG. 21A, the hole reamer 300 includes a cannulated shaft 302 defining a central lumen 304 extending longitudinally therethrough. As shown in FIG. 21B, the central lumen 304 of the hole reamer 300 defines a central axis 315 of the hole reamer 300. Furthermore, the cannulated shaft 302 has a first end 306 and a second end 308 that is provided at an opposite end of the cannulated shaft 302 from the first end 306. As shown in FIG. 21B, the central lumen 304 extends an entire length of the cannulated shaft 302 between the first end 306 and the second end 208.

Returning to FIG. 21A, the hole reamer 300 further includes a cutting end 310 provided at the first end 306 of the cannulated shaft 302. The cutting end 310 includes a plurality of cutting edges 314 extending along an outer surface 312 of the cutting end 310 and a counterbore 316 offset a distance from a distal end 318 of the cutting end 310. Shown best in FIG. 21D, the counterbore 316 is configured to abut an end surface of the bone to be shaped, thereby acting to limit the depth of the hole formed in the bone.

As shown in FIG. 21C, in the illustrated implementation, the plurality of cutting edges 314 includes three cutting edges 314. However, in further implementations, other numbers of cutting edges 314 may be included, so long as the hole reamer 300 is able to shape a hole into the end of a phalange. As shown, the cutting edges 314 are circumferentially arranged about the cutting end 310 of the peg reamer 300 cannulated shaft 302. Moreover, the plurality of cutting edges 314 are arranged circumferentially around the central axis 315 of the hole reamer 300. Specifically, the plurality of cutting edges 314 are evenly spaced around the central axis 315. Furthermore, as shown in FIG. 21D, the distal end 318 can taper radially inward toward the central axis 315.

As shown in FIGS. 21C-21E, the hole reamer 300 includes cutouts at the distal end 318 of the cutting end 310. These cutouts (best shown in FIG. 21E) can assist with removal of bone material from the targeted bone. As shown in FIG. 21C, the cutouts are circumferentially arranged about the central axis 315. Furthermore, as shown in FIG. 21E, the cutouts can have a radius of about 0.5 mm.

As shown in FIGS. 21D and 21G, the outer surface 312 of the cutting end 310 curves radially outward toward the counter bore 316. In some implementations, this radius of curvature ranges from 4.5 mm to 5.0 mm. In the illustrated implantation, this radius of curvature is about 4.75 mm. Thus, the cutting end 310 has a smooth profile, thereby forming a corresponding smooth profile within the target bone.

In accordance with the present disclosure, rotation of the plurality of cutting edges 314 about the central axis 315 of the hole reamer 300 defines a hole cutout region 322. This hole cutout region 322 corresponds to a hole formed in the target bone. In some implementations, the size and shape of the hole cutout region 322 can correspond to the size and shape of the bone material formed into a peg on the bone on the opposite side of the interphalangeal joint.

In some implementations, the hole cutout region 322 can have a depth ranging from 4.5 mm to 5.0 mm. In the implementation illustrated in FIGS. 21A-21F, the depth of the hole cutout region 322 is about 4.8 mm. In some implementations, the hole cutout region 322 can have a first diameter (D1) adjacent the counterbore 316 and a second diameter (D2) adjacent the distal end 318 of the hole reamer 300. In some implementations, the second diameter (D2) may range from 4.75 mm to 5.25 mm. In the implementation illustrated in FIGS. 21A-21F, the second diameter (D2) is about 5.1 mm. Accordingly, in the illustrated implementations, the plurality of cutting edges 314 slope inward relative to the central axis 315 from the counterbore 316 toward the distal end 318 of the hole reamer 300 such that the hole cutout region second diameter (D2) is larger than the hole cutout region first diameter (D1) and the hole cutout region is tapered toward the distal end 318. This ensures that the resulting hole formed in the patient's bone has a complementary taper. Furthermore, as provided herein, the taper of the hole cutout region 322 corresponds to the taper of the peg cutout region 224 such that the hole formed by the hole reamer 300 has a surface that complements a surface of the peg formed by the peg reamer 200. As shown in FIG. 21F, the cutting end 310 itself has an outer diameter of 12.0 mm.

In some implementations, the central lumen 304 of the hole reamer 300 can be sized and configured to receive the guide pin 400. In some implementations, the central lumen 304 of the hole reamer 300 can have a diameter ranging from 1.5 mm to 2.0 mm. As shown in FIG. 21B, in the illustrated implementation, the diameter of the central lumen 204 is about 1.8 mm.

As further described herein, the hole reamer 300 can be sized and configured to complement a corresponding counter-rotation ring 100, rescue dowel 500, and/or peg reamer 200. For example, the hole reamer 300 can be dimensioned to account for variations in anatomy and patient size. Accordingly, various implementations of the hole reamer 300 can have a range of dimensions, such as for accommodating both adult and pediatric patients.

The hole reamer 300 also includes a locking end 324 provided at the second end 308 of the cannulated shaft 302. The locking end 312 can be configured to engage a drill or other rotating device. The illustrated implementation further includes a flat 326 extending axially along the cannulated shaft 302 at the second end 308. The flat 326 can facilitate stable connection to a drill or other rotating device coupled to the second end 308. In the illustrated example, the flat 326 is best shown in FIGS. 21B and 21G.

In some implementations, the hole reamer 300 is formed from stainless steel. However, it is contemplated that the hole reamer 300 can be formed from any material that is capable of removing bone via rotation of the cutting end 310 and the cutting edges 314 thereon.

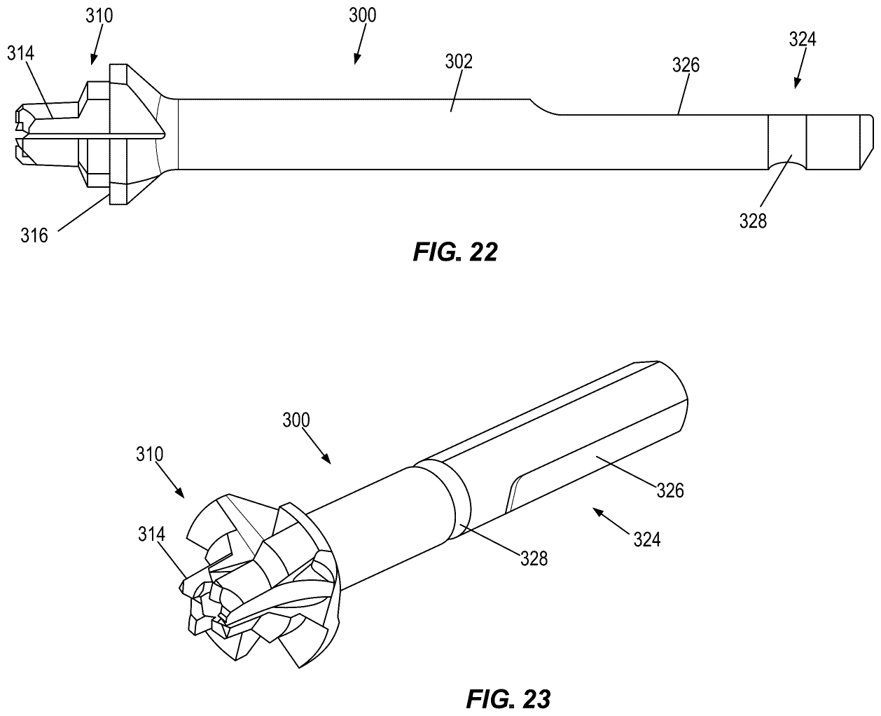

FIGS. 22-23 show various examples of the hole reamer 300. The hole reamers 300 in FIGS. 22-23 are similar to the hole reamer 300 described above in reference to FIGS. 21A-21G. Thus, similar reference numbers as those used for the implementation shown in FIGS. 21A-21G are used to reference similar features of the implementation shown in FIGS. 22-23. Furthermore, any features in any other implementations disclosed herein can be included in the implementation disclosed in FIGS. 21A-21G.

FIG. 22 shows a side view of an exemplary hole reamer 300 in which the locking end 212 further defines a groove 328 extending circumferentially around the cannulated shaft 302. This groove 328 can further facilitate formation of a stable connection to a drill or other rotating device coupled to the second end 308. For example, the groove 328 can itself provide a means of coupling the hole reamer 300 to a drill or other rotating device. FIG. 23 shows an implementation of the hole reamer 300 similar to the hole reamer 300 shown in FIG. 22. However, in this implementation, the groove 328 extends around the cannulated shaft 302 at an axial location distal to the flat 326.

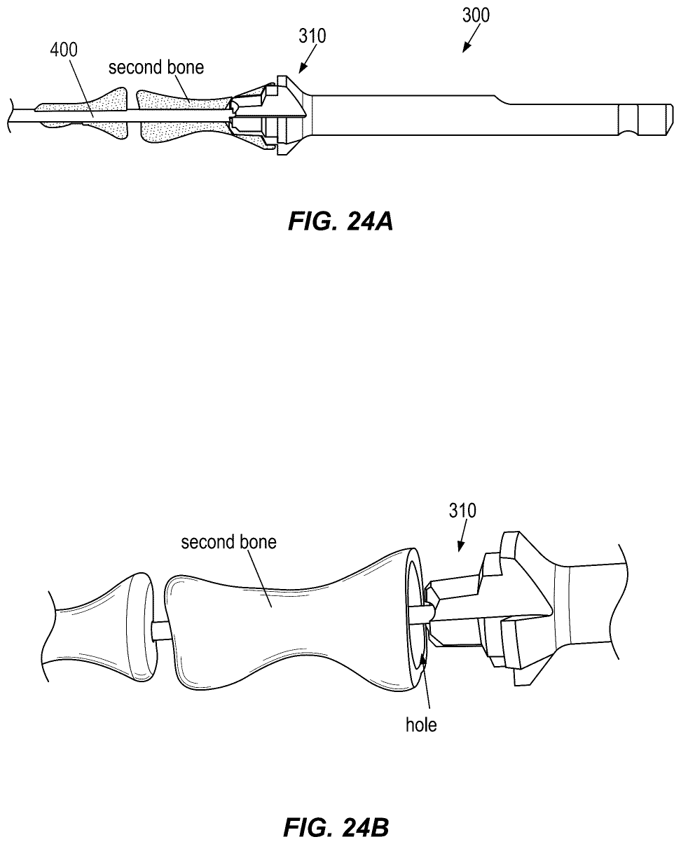

FIGS. 24A-24B show an exemplary hole reamer 300 shaping a bone. As shown, a guide pin 400 is advanced into a second bone along a longitudinal axis of the second bone. Once the guide pin 400 is placed, the hole reamer 300 is aligned with the guide pin 400 such that the central axis 315 of the hole reamer 300 is aligned with the longitudinal axis of a guide pin 400. The hole reamer 300 is then advanced over the guide pin 400 such that the guide pin 400 extends through the central lumen 304 of the hole reamer 300. In this way, the guide pin 400 establishes a path for the hole reamer 300 and defines the location of a subsequent hole formed in the second bone.

As shown in FIG. 24B, rotation of the cutting end 310 of the hole reamer 300 causes the cutting edges 314 to remove bone from a region of the first bone that is within the hole cutout region 322, thereby forming a hole in the second bone that corresponds to the shape of the hole cutout region 322. In the illustrated implementation, the second bone is a middle phalanx of a foot. However, in other implementations, the second bone could be another bone on either side of an interphalangeal joint. For example, the hole reamer 300 could be used to shape bone from a proximal phalanx of a foot. In further implementations, the hole reamer 300 could be used to shape bone on either side of an interphalangeal joint in a hand.

### Method of Joint Fusion

Provided herein is a method of fusing two bones on opposing sides of an interphalangeal joint using an interphalangeal joint fusion system. Such fusion allows for straightening of the phalanges on either side of the interphalangeal joint. As provided above, various reamers can be used to shape opposing ends of the two bones to be fused so that the two bones have complementary shapes. Accordingly, an interphalangeal joint fusion procedure begins by gaining access to the interphalangeal joint by separating the first and second bones on opposite sides of the joint and articulating the second bone relative to the first bone. As used herein, “first bone” refers to the bone on the proximal side of the interphalangeal joint and “second bone” refers to the bone on the distal side of the interphalangeal joint. With the joint-facing ends of the first and second bones exposed, a guide pin 400 can be inserted into the first bone. For example, the guide pin 400 can be advanced axially beginning at the inter-condylar notch at the sagittal midline. The guide pin 400 is advanced through the center of the medullary canal of the first bone until it reaches the base of the phalanx. In some implementations, channels that are sized and configured to receive the guide pin 400 can be formed through at least portions of the lengths of the phalanges on either side of the interphalangeal joint as well as other adjacent phalanges to facilitate insertion of the guide pin 400 therethrough. For example, the guide pin 400 can be inserted into a channel formed in the first bone.

As shown in FIG. 20A, the central axis 215 of the peg reamer 200 can then be aligned with the guide pin 400 and advanced over the guide pin 400 until the cutting end 210 of the peg reamer 200 is adjacent the joint-facing end of the first bone. In this way, the guide pin 400 can engage the central lumen 204 of the peg reamer 200. As shown in FIG. 20B, the cutting end 210 can then be rotated such that the cutting edges 216 remove bone material from the first bone until a peg of bone is formed that corresponds to the shape of the peg cutout region 224. In some implementations, the peg reamer 200 can be moved slightly back and forth in an axial direction to assist with bone removal until the peg reamer 200 cannot be advanced further (i.e., the peg of bone contacts a base of the peg cutout region 224). The peg reamer 200 and guide pin 400 can then be removed from the first bone.

In another aspect of the interphalangeal joint fusion procedure, another guide pin 400 can be inserted into the second bone in a retrograde direction. For example, the guide pin 400 can be inserted distally through the articular center surface of the phalanx, across the distal interphalangeal joint, and through the distal phalanx. As described above, in some implementations, channels that are sized and configured to receive the guide pin 400 can be formed through at least portions of the lengths of the phalanges on either side of the interphalangeal joint as well as other adjacent phalanges to facilitate insertion of the guide pin 400 therethrough. For example, the guide pin 400 can be inserted into a channel formed in the second and third bones.

As shown in FIG. 24A, the central axis 315 of the hole reamer 300 can then be aligned with the guide pin 400 and advanced over the guide pin 400 until the cutting end 310 of the hole reamer 300 is adjacent the joint-facing end of the second bone. In this way, the guide pin 400 can engage the central lumen 304 of the hole reamer 300. As shown in FIG. 24B, the cutting end 310 can then be rotated such that the cutting edges 314 remove bone material from the second bone until a hole is formed in the second bone that corresponds to shape of the hole cutout region 322. In some implementations, the hole reamer 300 can be moved slightly back and forth in an axial direction to assist with bone removal until the hole reamer 300 cannot be advanced further (i.e., the counterbore 316 contacts the second bone). In some implementations, the surgical site can be irrigated during the procedure. Any residual bone or tissue at the articular surface of the phalanx is removed. The hole reamer 300 can then be removed from the guide pin 400. While the above description provides for formation of the peg of bone prior to formation of the hole, it is contemplated herein, that either the peg of bone or hole can be formed first.

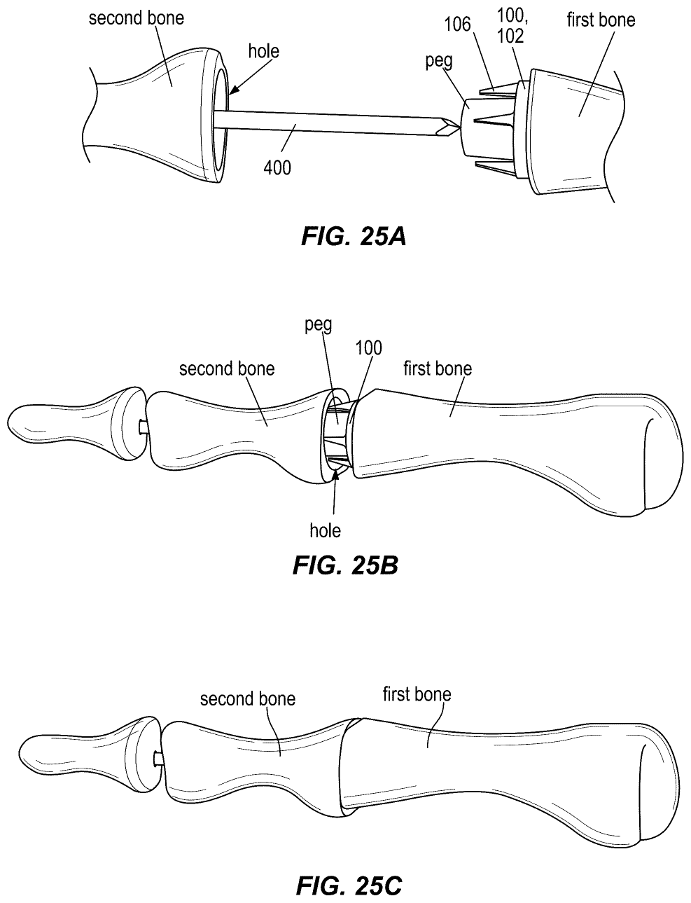

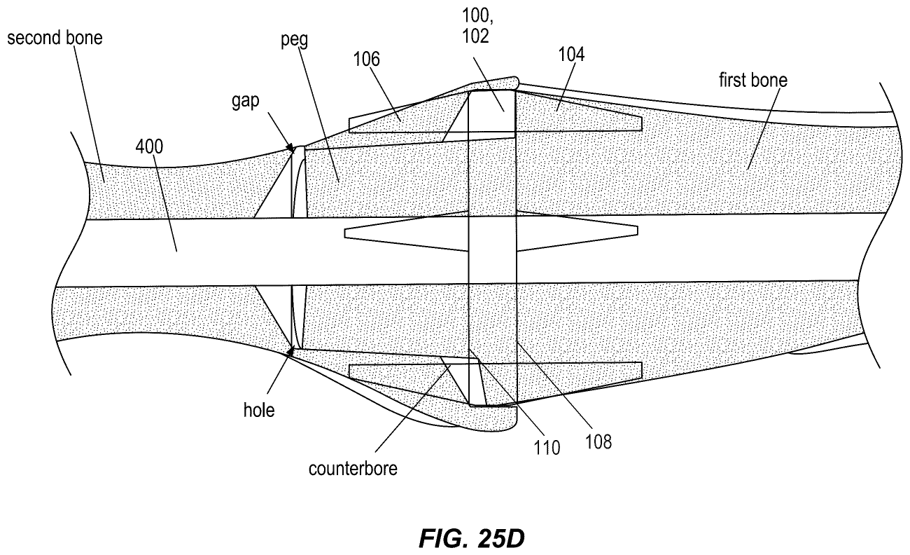

Once the peg of bone and an opposing hole are formed in the respective first and second bones, a counter-rotation ring 100 is inserted into the first bone. Specifically, as shown in FIG. 25A, proximal axial spikes 104 (obscured by the first bone) are inserted into the first bone such that the proximal surface 108 of the annular body 102 abuts a shelf-like surface formed in the joint-facing end of the first bone. As shown, annular body 102 of the counter-rotation ring 100 extends around the peg of bone. In other words, the peg of bone extends axially through the central lumen 126 of the counter-rotation ring 100. Once the counter-rotation ring 100 is anchored in the first bone, the second bone is aligned with the first bone so that longitudinal axes of the two bones are aligned. As shown in FIGS. 25C and 25D, the first and second bones are moved toward each other along their longitudinal axes. Specifically, as shown in FIG. 25B, the guide pin 400 disposed within the second bone is inserted into the peg of bone and the second bone is advanced toward the first bone so that the distal axial spikes 106 of the counter-rotation ring 100 are inserted into the second bone such that the distal surface 110 of the annular body abuts a shelf-like surface formed around the hole in the joint-facing end of the second bone. In some implementations, the shelf-like surface formed in the second bone is produced by the counterbore 316 of the hole reamer 300. As shown in FIGS. 25C-25D, the first bone and the second bone can then be placed in an assembled relationship in which the peg of bone on the first bone is received within the opposing hole defined in the second bone. In this way, the first bone and second bone are held in contact, thereby facilitating fusion of the interphalangeal joint. In some implementations, the peg of bone on the first bone and the hole in the second bone frictionally engage so as to form an interference fit, thereby increasing the surface area over which the bones can fuse. Furthermore, as described herein, the counter-rotation ring 100 rotationally fixes the first bone and the second bone, thereby facilitating fusion of the assembled first and second bones. It is contemplated herein that the interphalangeal joint fusion system can be used to stabilize bones on opposing sides of an interphalangeal joint so as to aid in the fixation of fractures, fusions, and osteotomies of phalanges.

FIG. 25D shows a partial cross-sectional view of the first and second bones brought into contact with the counter-rotation ring 100 disposed therebetween. As shown, the annular ring 102 is disposed within a region defined by the counterbore 316 such that the proximal surface 108 is in contact with the first bone and the distal end 310 is in contact with the second bone. FIG. 25D shows the peg of bone of the first bone being received within the hole of the second bone such that surfaces of the peg of bone and hole are in direct contact. This facilitates subsequent fusion of the first and second bone. In some implementations, as illustrated in FIG. 25D, a small gap can exist between a distal end of the peg of bone and a base of the hole. Such a gap can help ensure that the surfaces of the first and second bones on either side of the counter-rotation ring 100 are in flush contact with the respective proximal surface 108 and distal surface 110 of the counter-rotation ring 100.

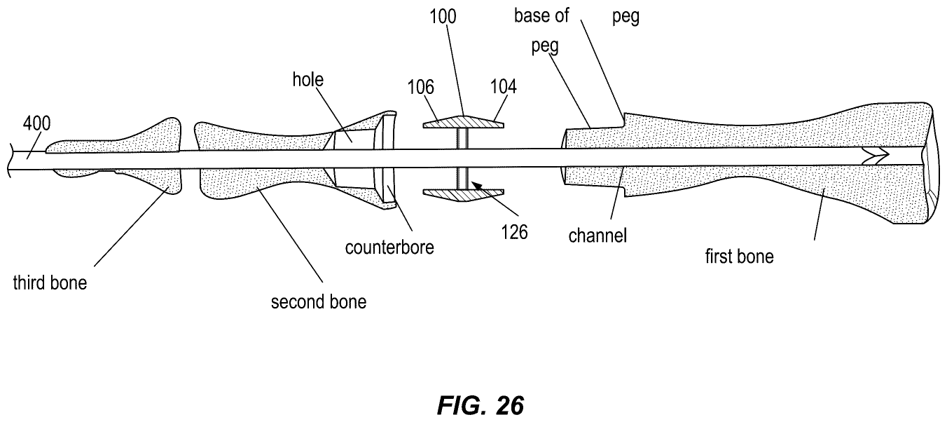

FIG. 26 shows an exploded side view of the exemplary interphalangeal joint fusion system shown in FIGS. 25A-25D, including the positions of the counter-rotation ring 100 guide pin 400 positioned relative to the first and second bones of a patient's foot.

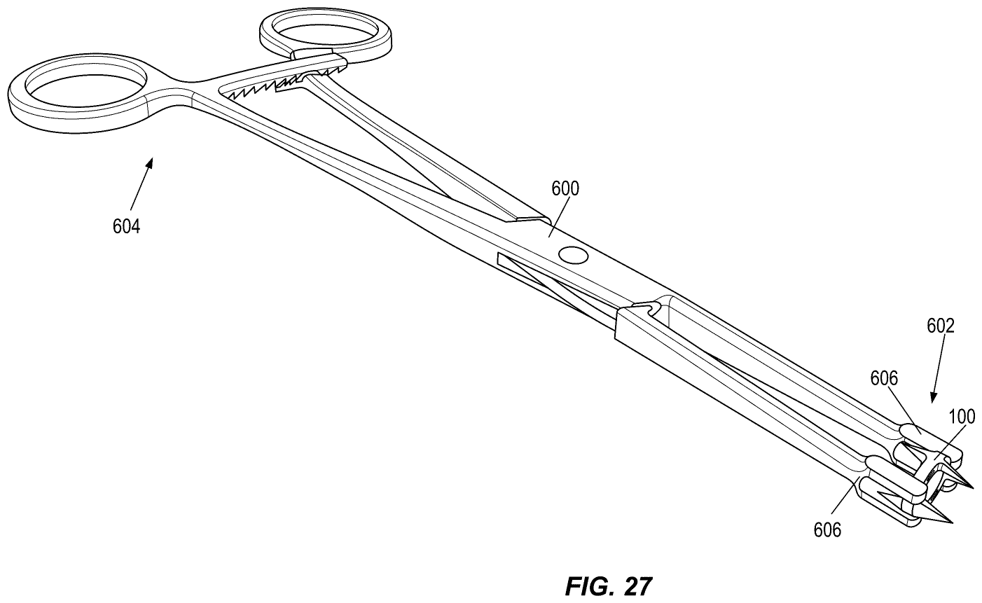

FIG. 27 shows an exemplary gripping tool 600 gripping an exemplary counter-rotation ring 100. As shown in FIG. 27, the gripping tool 600 includes a grasping end 602 for grasping a counter-rotation ring 100 and a gripping end 604 opposite the grasping end 602 for manipulating the gripping tool 600. The grasping end 602 includes forked arms 606 that are sized and configured to grip corresponding geometries (for example, the staple cutouts 142) of the counter-rotation ring 100.

### Rescue Dowel

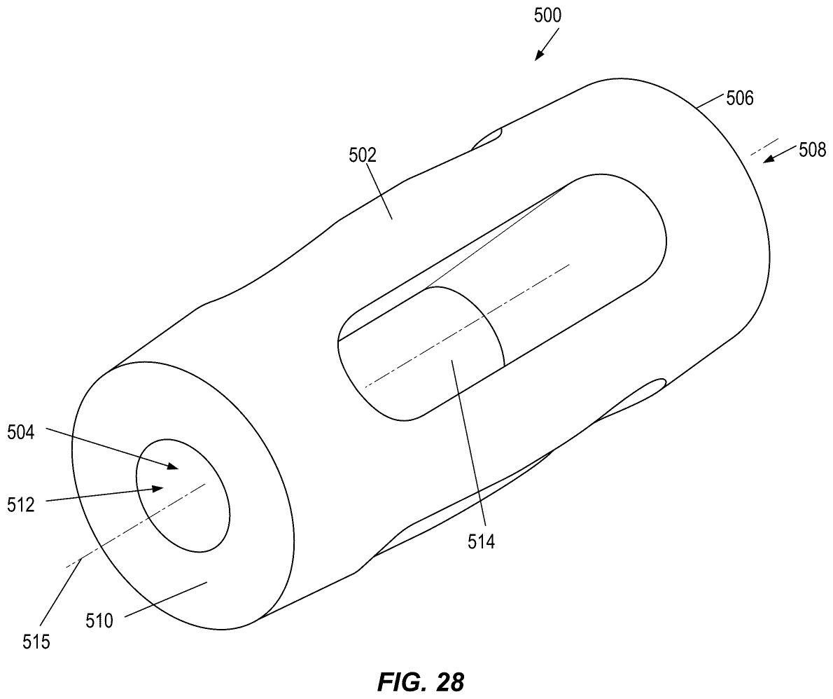

In some instances, it is possible that the region of the first bone intended to be formed into the peg of bone is sheared off. In such circumstances, an alternative structure is needed to anchor the first bone into the hole formed in the second bone. Accordingly, in some implementations, the interphalangeal joint fusion system can further include a rescue dowel 500 (also referred to as a “recovery dowel”). As shown in FIG. 28, the rescue dowel 500 includes a tubular body 502 and defines a central lumen 504 extending longitudinally therethrough. The tubular body 502 has a proximal end 506 defining a proximal end opening 508 that extends to the central lumen 504 and a distal end 510 defining a distal end opening 512 that extends to the central lumen 504. In some implementations, the central lumen 504, proximal end opening 508, and distal end opening 512 are sized and configured to receive a guide pin 400. The central lumen 504 also defines a central axis 515 extending longitudinally therethrough.

In some implementations, the proximal end 506 and distal end 510 can be sized and configured to frictionally engage the hole defined in the second bone. Accordingly, the proximal end 506 and distal end 510 are sized to match the hole cutout region 322. As shown in the implementation illustrated in FIG. 28, the tubular body 502 further defines a plurality of axial slots 514 arranged circumferentially about the tubular body 502 and extending inward toward the central lumen 504. Specifically, the plurality of axial slots 514 are spaced evenly about a circumference of the tubular body 502. In some implementations, the axial slots are configured to allow bone ingrowth into the central lumen 504, thereby improving fusion of the first and second bones. Furthermore, implantation of a rescue dowel 500 can help preserve the length of the patient's interphalangeal joint in instances of end-to-end fusion.

As further described herein, the rescue dowel 500 can be sized and configured to correspond with the central lumen 126 of a counter-rotation ring 100. In further implementations, the rescue dowel 500 can be dimensioned to account for variations in anatomy and patient size. Accordingly, various implementations of the rescue dowel 500 can have a range of dimensions, such as for accommodating both adult and pediatric patients.

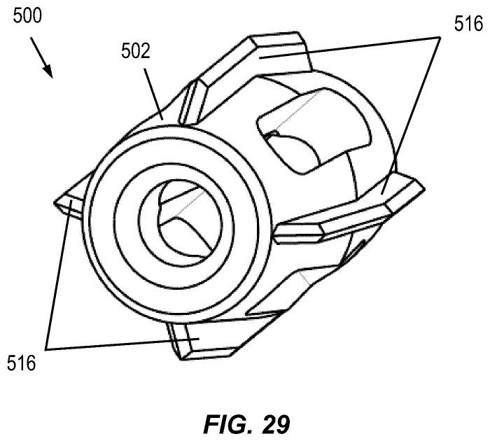

FIGS. 29-32B show various examples of the rescue dowel 500. The rescue dowels 500 in FIGS. 29-32B are similar to the rescue dowel 500 described above in reference to FIG. 28. Thus, similar reference numbers as those used for the implementation shown in FIGS. 29-32B are used to reference similar features of the implementation shown in FIG. 28. Furthermore, any features in any other implementations disclosed herein can be included in the implementation disclosed in FIG. 28.

FIG. 29 shows an exemplary rescue dowel 500 similar to the rescue dowel described above with respect to FIG. 28. However, the exemplary rescue dowel 500 illustrated in FIG. 29 further comprises fins 516 extending radially from an outer surface of the tubular body 502. Specifically, in the illustrated implementation, the axial fins 516 are arranged circumferentially about the tubular body 502 and are spaced evenly about a circumference of the tubular body 502. Furthermore, as shown, the axial fins 516 extend between the proximal end 506 and the distal end 510. In some implementations, the axial fins 516 improve anchoring between the rescue dowel 500 and the first and/or second bones. Furthermore, the axial fins 516 can provide further resistance to relative rotational movement of the first and second bones.

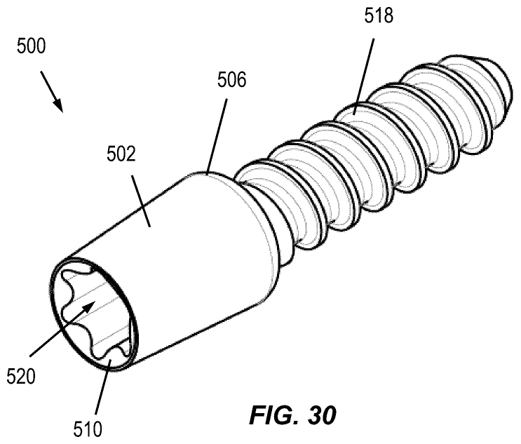

FIG. 30 shows an exemplary rescue dowel 500 similar to the rescue dowel described above in reference to FIG. 28. However, the exemplary rescue dowel 500 illustrated in FIG. 30 further comprises a threaded shaft extending axially from the proximal end 506 the tubular body 502. As shown in FIG. 30, the illustrated implementation includes a shaped recess 520 defined within the distal end 510 of the rescue dowel. In some implementations, the shaped recess 520 is sized and configured to correspond to a driver used to rotate the rescue dowel 500, thereby facilitating implantation of the rescue dowel 500 into a bone.

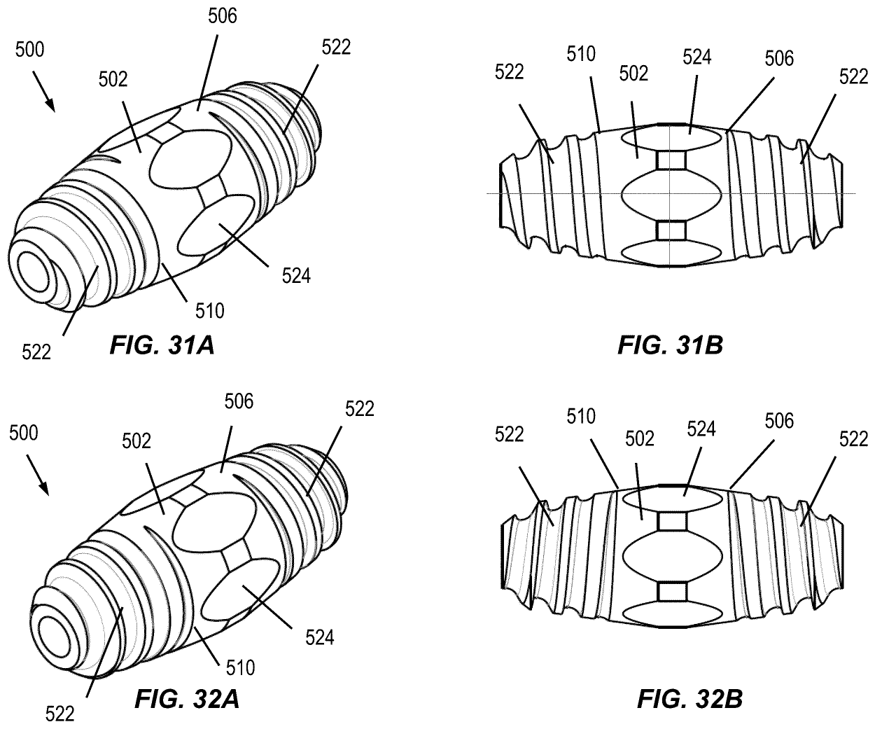

FIGS. 31A and 31B show an exemplary rescue dowel 500 similar to the rescue dowel described above in reference to FIG. 28. However, in this implementation, the proximal and distal ends 506, 510 include externally threaded surfaces. Specifically, in the illustrated implementation, the threaded surfaces are unidirectional. Furthermore, as shown in FIGS. 31A and 31B, the rescue dowel 500 does not contain axial slots 514. Instead, flats 524 are arranged about the circumference of the tubular body 502. These flats 524 can facilitate gripping of the rescue dowel 500 for the purpose of threading the rescue dowel 500 into a first or second bone.

FIGS. 32A and 32B show an exemplary rescue dowel 500 similar to the rescue dowel described above in reference to FIGS. 31A and 31B. However, in this implementation, the externally threaded surfaces on the proximal and distal ends 506, 510 are threaded in opposite directions. This can help ease placement of the rescue dowel 500, as the first and second bones can be held rotationally relative to each other and the rescue dowel 500 can be rotated relative to both the first and second bones via the flats 524.

The rescue dowel 500 can be formed from various biocompatible materials. For example, in some implementations, the counter-rescue dowel 500 can be formed from a polymer, including resorbable and non-resorbable polymers as well as co-polymers. In further implementations, rescue dowel 500 can be formed from a metal, such as titanium and/or a titanium alloy.

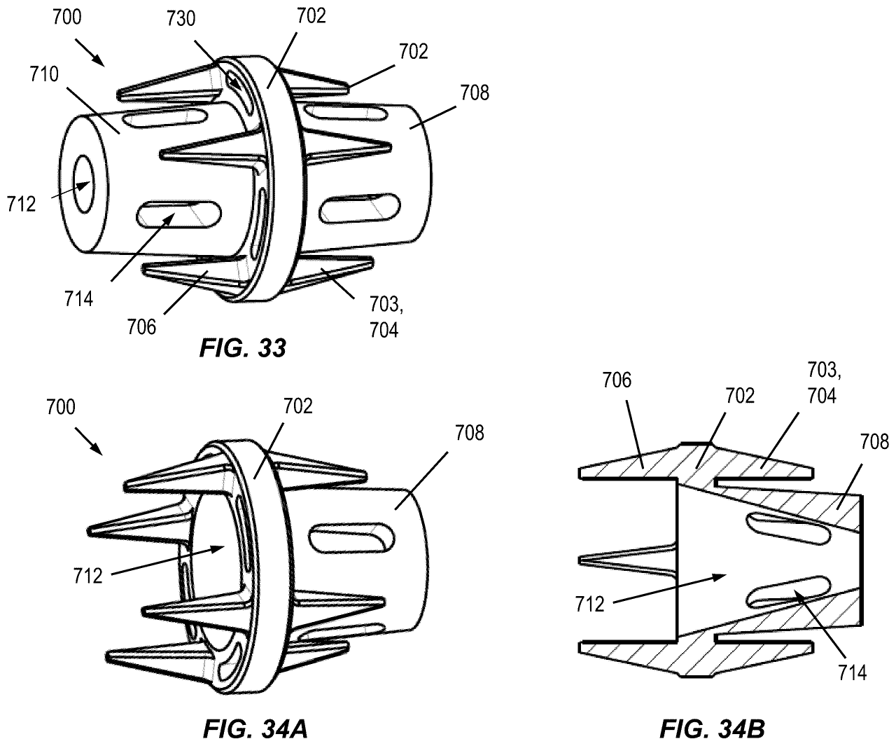

In some implementations, the interphalangeal joint fusion system can include a combined counter-rotation ring and rescue dowel 700. As illustrated in FIG. 33, the combined counter-rotation ring and rescue dowel 700 includes an annular body 702 similar to the annular body 102 described above in reference to the counter-rotation ring 100 shown in FIG. 1A. For example, the annular body 702 includes axial spikes 703. Specifically, as shown in FIG. 33, the annular body 702 includes a plurality of proximal axial spikes 704 that are similar to the proximal axial spikes 104 and distal axial spikes 706 that are similar to the distal axial spikes 106. Additionally, as shown in FIG. 33, the annular body 702 includes a plurality of circumferential slots 730 similar to circumferential slots 130.

Furthermore, the combined counter-rotation ring and rescue dowel 700 includes a tubular body similar to the tubular body 502 described above in reference to the rescue dowel 500 shown in FIG. 28. Specifically, tubular body is divided into a proximal tubular body 708 that extends proximally from the annular body 702 and a distal tubular body 710 that extends distally form the annular body 702. The annular body proximal and distal tubular bodies 708, 710 define a central lumen 712 extending longitudinally through the combined counter-rotation ring and rescue dowel 700. Additionally, as shown in FIG. 33, a plurality axial slots 714 circumferentially arranged about the proximal and distal tubular bodies 708, 710 that are similar to the axial slots 514.

FIGS. 34A and 34B show an exemplary combined counter-rotation ring and rescue dowel 700 similar to the combined counter-rotation ring and rescue dowel described above in reference to FIG. 33. However, in this implementation, the tubular body extends from only one side of the annular body 702. Specifically, in the implementation illustrated in FIG. 34A, a proximal tubular body 708 extends proximally from the annular body 702 while no corresponding tubular body extends in an opposite direction. Furthermore, as shown in FIG. 34B, in this implementation, the central lumen 712 narrows from a wider diameter at the annular body 702 to a progressively narrower diameter moving along the inner surface of the proximal tubular body 708. While the illustrated implementation shows the combined counter-rotation ring and rescue dowel 700 having a proximal tubular body 708 and no distal tubular body 710, in some implementations, the combined counter-rotation ring and rescue dowel 700 having a distal tubular body 710 and no proximal tubular body 708.

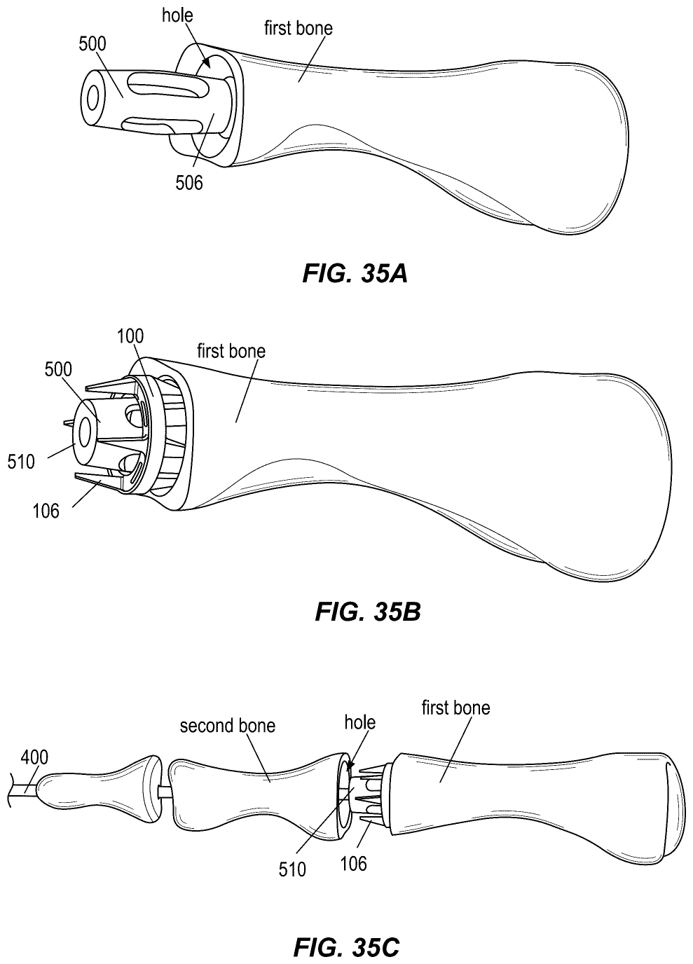

In instances where the peg of bone is sheared off, the above-described method for fusing first and second bones on opposing sides of an interphalangeal joint can be modified. For example, FIGS. 35A-35C show a method of using a rescue dowel 500 as an alternative to formation of a peg of bone in the first bone. As shown in FIGS. 35A-35C, rather than using a peg reamer 200 to form a peg of bone in the first bone, a hole reamer 300 is used in a manner similar to that described above with reference to the second bone. Specifically, instead of advancing a peg reamer 200 toward the first bone along guide pin 400, the central axis 315 of the hole reamer 300 can be aligned with the guide pin 400 and advanced over the guide pin 400 until the cutting end 310 of the hole reamer 300 is adjacent the joint-facing end of the first bone. The cutting end 310 can then be rotated such that the cutting edges 314 remove bone material from the first bone until a hole is formed in the first bone that corresponds to shape of the hole cutout region 322. Thus, the method involves forming opposing holes in the first and second bones on both sides of the interphalangeal joint.

As shown in FIG. 35A, the proximal end 506 of the tubular body 502 of a rescue dowel 500 can then be inserted into the hole formed in the first bone. In some implementations, inserting the rescue dowel 500 into the hole formed in the first bone includes advancing the rescue dowel 500 over the guide pin 400 such that the guide pin 400 is received within the central lumen 504 of the tubular body 502.

Once the rescue dowel 500 is anchored within the hole formed in the first bone, the counter-rotation ring 100 can then be inserted into the first bone. As shown in FIG. 35B, when the proximal axial spikes 104 of the counter-rotation ring 100 are inserted into the first bone and the annular body 102 of the counter-rotation ring 100 is advanced toward the first bone so that it abuts the first bone, the annular body 102 of the counter-rotation ring 100 extends around the tubular body 502 of the rescue dowel 500. In other words, the rescue dowel 500 extends axially through the central lumen 126 of the counter-rotation ring 100. Furthermore, as shown in the implementation illustrated in FIG. 35B, the proximal surface 108 of the annular body 102 abuts a shelf-like surface surrounding the hole of the first bone that is formed by the counterbore 316 of the hole reamer 300.

Once the counter-rotation ring 100 is anchored in the first bone, the second bone is aligned with the first bone so that longitudinal axes of the two bones are aligned. As shown in FIG. 35C, the first and second bones are moved toward each other along their longitudinal axes. Specifically, as shown in FIG. 35C, the guide pin 400 disposed within the second bone is inserted into the central lumen 504 of the tubular body 502 and the second bone is advanced toward the first bone so that the distal axial spikes 106 of the counter-rotation ring 100 are inserted into the second bone such that the distal surface 110 of the annular body abuts a shelf-like surface formed around the hole in the joint-facing end of the second bone. In some implementations, the guide pin 400 can engage the central lumen 504 of the rescue dowel 500.

As described herein, in some implementations, the shelf-like surface formed in the second bone is produced by the counterbore 316 of the hole reamer 300. As shown in FIG. 35C, the first bone and the second bone can then be placed in an assembled relationship in which the distal end 510 of the tubular body 502 of the rescue dowel 500 is received within the hole defined in the second bone. In this way, the first bone and second bone are held in contact, thereby facilitating fusion of the interphalangeal joint. In some implementations, the rescue dowel 500 frictionally engages each respective hole in the first bone and second bone so as to form respective interference fits, thereby increasing stability of the joint and promoting fusion of the first and second bones.

It is contemplated herein that any combination of the above-described counter-rotation ring 100, peg reamer 200, hole reamer 300, guide pin 400, rescue dowel 500, and/or combined counter-rotation ring and rescue dowel 700 can be used to perform variations of the interphalangeal joint fusion methods described herein. For example, steps in the procedures described above can be performed in other orders or omitted entirely, depending on the treatment needs of a particular patient. Furthermore, while various implementations of the interphalangeal joint fusion provided herein are described in reference to interphalangeal joint fusion of the proximal and middle phalanxes of the foot for treatment of hammertoe, it is contemplated that the systems and methods described herein may be applicable to other anatomies. For example, the interphalangeal joint fusion systems and methods provided herein can be used to perform fusion of proximal and middle phalanges of the hand.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An interphalangeal joint fusion system comprising: a counter-rotation ring comprising an annular body including a proximal spike extending axially from a proximal surface of the annular body and a distal spike extending axially from a distal surface of the annular body.

Example 2: The interphalangeal joint fusion system according to any example herein, particularly example 1, wherein the proximal spike includes a plurality of proximal spikes extending axially from the proximal surface of the annular body and the distal spike includes a plurality of distal spikes extending axially from the distal surface of the annular body.

Example 3: The interphalangeal joint fusion system according to any example herein, particularly example 2, wherein the plurality of proximal spikes are arranged circumferentially about the proximal surface of the annular body, and the plurality of distal spikes are arranged circumferentially about the distal surface of the annular body.

Example 4: The interphalangeal joint fusion system according to any example herein, particularly examples 2-3, wherein the plurality of proximal spikes and the plurality of distal spikes are evenly spaced about the corresponding proximal surface and distal surface.

Example 5: The interphalangeal joint fusion system according to any example herein, particularly examples 2-4, wherein a circumferential location of each of the plurality of proximal spikes on the proximal surface corresponds with a circumferential location of a corresponding one of the plurality of distal spikes on the distal surface.

Example 6: The interphalangeal joint fusion system according to any example herein, particularly examples 1-5, wherein at least one of the proximal spike or the distal spike has a decreasing width from the corresponding proximal surface or distal surface of the annular body toward an axial spike tip of the proximal spike or the distal spike.

Example 7: The interphalangeal joint fusion system according to any example herein, particularly examples 1-6, wherein at least one of the proximal spike or distal spike includes an inner surface that is angled radially inward toward a central axis of the annular body.

Example 8: The interphalangeal joint fusion system according to any example herein, particularly example 7, wherein at least one of the proximal spike or distal spike includes a barb extending radially inward from an inner surface of the spike toward a central axis of the annular body.

Example 9: The interphalangeal joint fusion system according to any example herein, particularly examples 7-8, wherein at least one of the proximal spike or distal spike includes an outer surface extending generally parallel with a central axis of the annular body.

Example 10: The interphalangeal joint fusion system according to any example herein, particularly examples 1-7, wherein at least one of the proximal spike or distal spike includes an inner surface extending generally parallel with a central axis of the annular body, and an outer surface having a decreasing taper from the corresponding proximal surface or distal surface of the annular body toward a spike tip of the proximal spike or distal spike.

Example 11: The interphalangeal joint fusion system according to any example herein, particularly examples 1-10, wherein at least one of the proximal spike or distal spike includes a barb extending radially outward from an outer surface of the spike away from a central axis of the annular body.

Example 12: The interphalangeal joint fusion system according to any example herein, particularly examples 1-11, wherein the annular body includes a plurality of circumferential slots arranged circumferentially about the annular body and extending at least partially through the annular body.

Example 13: The interphalangeal joint fusion system according to any example herein, particularly examples 12, wherein at least one of the plurality of circumferential slots extends through the annular body from the proximal surface to the distal surface.

Example 14: The interphalangeal joint fusion system according to any example herein, particularly examples 12-13, wherein the plurality of circumferential slots are spaced evenly about a circumference of the annular body.

Example 15: The interphalangeal joint fusion system according to any example herein, particularly examples 12-14, wherein the plurality of circumferential slots include an elongated body portion having a curvature corresponding to a curvature of the annular body.

Example 16: The interphalangeal joint fusion system according to any example herein, particularly examples 1-15, wherein the annular body includes a suture opening extending through the annular body.

Example 17: The interphalangeal joint fusion system according to any example herein, particularly examples 1-16, wherein the annular body includes a staple cutout extending radially inward from an outer surface of the annular body toward a central axis of the annular body.

Example 18: The interphalangeal joint fusion system according to any example herein, particularly examples 1-17, wherein the annular body includes a central lumen extending therethrough, an inner surface of the central lumen including a radial projection extending radially away from a central axis of the annular body.

Example 19: The interphalangeal joint fusion system according to any example herein, particularly examples 1-18, wherein the annular body includes a central lumen extending therethrough, the central lumen having an inner diameter ranging from 4.0 mm to 5.0 mm, and the annular member having an outer diameter ranging from 6 mm to 7 mm.

Example 20: The interphalangeal joint fusion system according to any example herein, particularly examples 1-19, wherein the annular body has a thickness ranging from 0.5 mm to 1.5 mm.

Example 21: The interphalangeal joint fusion system according to any example herein, particularly examples 1-20, wherein the annular body has a generally circular-shaped outer surface and a generally rectangular-shaped cross-section.

Example 22: The interphalangeal joint fusion system according to any example herein, particularly examples 1-21, further including a peg reamer comprising: a cannulated shaft defining a central lumen extending therethrough; a cutting end provided at a first end of the cannulated shaft, the cutting end including a plurality of cutting arms extending from the cannulated shaft, each cutting arm includes a cutting edge extending along an inner surface of each cutting arm; and a locking end provided at an opposing second end of the cannulated shaft from the cutting end.

Example 23: The interphalangeal joint fusion system according to any example herein, particularly example 22, wherein the central lumen of the peg reamer cannulated shaft defines a central axis of the peg reamer, where and each of the cutting edges are spaced apart from the central axis of the cannulated shaft.

Example 24: The interphalangeal joint fusion system according to any example herein, particularly example 23, wherein a spacing between each of the cutting edges and the central axis of the peg reamer cannulated shaft ranges from 2.25 mm to 2.75 mm.

Example 25: The interphalangeal joint fusion system according to any example herein, particularly examples 22-24, wherein the plurality of cutting arms are circumferentially arranged about the cutting end of the peg reamer cannulated shaft.

Example 26: The interphalangeal joint fusion system according to any example herein, particularly examples 22-25, wherein rotation of the plurality of cutting arms about a central axis of the peg reamer defines a peg cutout region therebetween.

Example 27: The interphalangeal joint fusion system according to any example herein, particularly example 26, wherein the peg cutout region has a depth ranging from 4.5 mm to 5.5 mm.

Example 28. The system of any one of examples 26-27, wherein the peg cutout region has a first diameter adjacent a proximal end of the peg cutout region and a larger second diameter adjacent a distal end of the peg cutout region.

Example 29: The interphalangeal joint fusion system according to any example herein, particularly examples 26-28, wherein the cutting edges slope outward relative to a central axis of the peg reamer toward a distal end of the cutting arms, wherein the peg cutout region second diameter is larger than the peg cutout region first diameter such that the peg cutout region is tapered.

Example 30: The interphalangeal joint fusion system according to any example herein, particularly examples 22-29, wherein the central lumen of the peg reamer is sized and configured to receive a guide pin.

Example 31: The interphalangeal joint fusion system according to any example herein, particularly examples 22-30, wherein the central lumen of the peg reamer has a diameter ranging from 1.5 mm to 2.0 mm.

Example 32: The interphalangeal joint fusion system according to any example herein, particularly examples 22-31, wherein the plurality of cutting arms comprises four cutting arms.

Example 33: The interphalangeal joint fusion system according to any example herein, particularly examples 22-32, wherein the locking end of the peg reamer includes a flat extending axially along the cannulated shaft.

Example 34: The interphalangeal joint fusion system according to any example herein, particularly examples 22-33, wherein the locking end of the peg reamer comprises a groove extending circumferentially around the cannulated shaft.

Example 35: The interphalangeal joint fusion system according to any example herein, particularly examples 1-34, further including a hole reamer comprising: a cannulated shaft defining a central lumen extending therethrough; a cutting end provided at a first end of the cannulated shaft, the cutting end including a plurality of cutting edges extending along an outer surface of the cutting end, and a counterbore offset a distance from a distal end of the cutting end; and a locking end provided at an opposing second end of the cannulated shaft from the cutting end.

Example 36: The interphalangeal joint fusion system according to any example herein, particularly example 35, wherein the central lumen of the hole reamer cannulated shaft defines a central axis of the hole reamer, wherein rotation of the plurality of cutting edges about the hole reamer central axis defines a hole cutout region.

Example 37: The interphalangeal joint fusion system according to any example herein, particularly example 36, wherein the hole cutout region has a depth ranging from 4.5 mm to 5.0 mm.

Example 38: The interphalangeal joint fusion system according to any example herein, particularly examples 36-37, wherein the hole cutout region has a first diameter adjacent the counterbore and a second diameter adjacent the distal end of the hole reamer.

Example 39: The interphalangeal joint fusion system according to any example herein, particularly examples 37-38, wherein the plurality of cutting edges slope inward relative to the central axis from the counterbore toward the distal end of the hole reamer such that the hole cutout region second diameter is larger than the hole cutout region first diameter and the hole cutout region is tapered toward the distal end.

Example 40: The interphalangeal joint fusion system according to any example herein, particularly examples 36-39, wherein the central lumen of the hole reamer is sized and configured to receive a guide pin.

Example 41: The interphalangeal joint fusion system according to any example herein, particularly examples 36-40, wherein the central lumen of the hole reamer cannulated shaft has a diameter ranging from 1.5 mm to 2.0 mm.

Example 42: The interphalangeal joint fusion system according to any example herein, particularly examples 36-41, wherein the locking end of the hole reamer cannulated shaft comprises a flat.

Example 43: The interphalangeal joint fusion system according to any example herein, particularly examples 36-42, wherein the locking end of the hole reamer cannulated shaft comprises a groove.

Example 44: The interphalangeal joint fusion system according to any example herein, particularly examples 1-43, further including a rescue dowel comprising: a tubular body defining a central lumen extending therethrough, the tubular body having a proximal end defining a proximal end opening extending to the central lumen and a distal end defining a distal end opening extending to the central lumen.

Example 45: The interphalangeal joint fusion system according to any example herein, particularly examples 35-44, wherein the proximal end and distal end are sized and configured to frictionally engage with the hole cutout region.

Example 46: The interphalangeal joint fusion system according to any example herein, particularly examples 44-45, wherein the tubular body further defines a plurality of axial slots arranged circumferentially about the tubular body and extending toward the central lumen.

Example 47: The interphalangeal joint fusion system according to any example herein, particularly example 46, wherein the plurality of axial slots are spaced evenly about a circumference of the tubular body.

Example 48: The interphalangeal joint fusion system according to any example herein, particularly examples 44-47, wherein the central lumen of the rescue dowel is sized and configured to receive a guide pin.

Example 49: The interphalangeal joint fusion system according to any example herein, particularly examples 1-48, further including a guide pin sized and configured to extend from and frictionally engage with a distal bone of a joint through at least a portion of a proximal bone of the joint, wherein the guide pin is further sized and configured to be received within the central lumen of the peg reamer and the central lumen of the hole reamer.

Example 50: The interphalangeal joint fusion system according to any example herein, particularly example 49, wherein the guide pin is sized and configured to be received within the central lumen of the rescue dowel.

Example 51: A kit for the surgical treatment of deformity of the interphalangeal joints, the kit comprising: a counter-rotation ring comprising an annular body including a proximal spike extending axially from a proximal surface of the annular body and a distal spike extending axially from a distal surface of the annular body; a peg reamer comprising: a cannulated shaft defining a central lumen extending therethrough; a cutting end provided at a first end of the cannulated shaft, the cutting end including a plurality of cutting arms extending from the cannulated shaft, each of the plurality of cutting arms includes a cutting edge extending along an inner surface of the corresponding cutting arm; and a locking end provided at an opposing second send of the cannulated shaft from the cutting end; a hole reamer comprising: a cannulated shaft defining a central lumen extending therethrough; a cutting end provided at a first end of the cannulated shaft, the cutting end including a plurality of cutting edges extending along an outer surface of the cutting end, and a counterbore offset a distance from a distal end of the cutting end; and a locking end provided at an opposing second end of the cannulated shaft from the cutting end.

Example 52: The kit for the surgical treatment of deformity of the interphalangeal joints according to any example herein, particularly example 51, further including a rescue dowel comprising: a tubular body defining a central lumen extending therethrough, the tubular body having a proximal end defining a proximal end opening extending to the central lumen and a distal end defining a distal end opening extending to the central lumen.

Example 53: The kit for the surgical treatment of deformity of the interphalangeal joints according to any example herein, particularly examples 51-52, further including a guide pin sized and configured to extend from and frictionally engage with a phalange on a distal side of a patient's joint through at least a portion of a phalange on a proximal side of the patient's joint, wherein the guide pin is further sized and configured to be received within central lumen of the peg reamer and the central lumen of the hole reamer.

Example 54. A method for straightening an interphalangeal joint, comprising: accessing an interphalangeal joint; separating first and second bones on opposite sides of the interphalangeal joint; removing material from one of the first or second bone so as to form a peg and removing material from the other bone so as to form an opposing hole, wherein the peg is sized and configured to be received by and frictionally engage with the hole; positioning a counter-rotation ring around the peg, wherein the counter-rotation ring comprises an annular body having a first surface and an opposing second surface, wherein a plurality of axial spikes extend from at least one of the first and second surfaces; inserting at least one axial spike into the first or second bone from which the peg is formed such that the first surface of the annular body abuts a surface that surrounds the peg; placing the first and second bones in an assembled relationship such that the hole receives the peg; inserting at least one axial spike into the first or second bone from which the hole is formed such that the second surface of the annular body abuts a surface that surrounds the hole; wherein the counter-rotation ring rotationally fixes the first bone and second bone, thereby facilitating fusion of the assembled first and second bones.

Example 55: The method for straightening an interphalangeal joint according to any example herein, particularly example 54, further comprising defining channels through at least portions of the lengths of both the first and second bones, wherein the channels are sized and configured to receive a guide pin therethrough.

Example 56: The method for straightening an interphalangeal joint according to any example herein, particularly example 55, further comprising introducing the guide pin into the channel of the first bone.

Example 57: The method for straightening an interphalangeal joint according to any example herein, particularly examples 55 or 56, further comprising introducing the guide pin into the channel of the second bone.

Example 58: The method for straightening an interphalangeal joint according to any example herein, particularly examples 56 or 57, wherein the step of removing material from one of the first or second bone so as to form a peg further comprises engaging the guide pin with a central lumen of a peg reamer.

Example 59: The method for straightening an interphalangeal joint according to any example herein, particularly example 58, wherein the step of removing material from the other of the first or second bone so as to form an opposing hole further engaging the guide pin with a central lumen of a hole reamer.

Example 60. A method for straightening an interphalangeal joint, comprising: accessing an interphalangeal joint; separating bones on opposite sides of the interphalangeal joint, wherein a first bone is proximal relative to the joint and a second bone is distal relative to the joint; removing material from one of the first or second bone so as to form a hole and removing material from the other bone so as to form an opposing hole; inserting a rescue dowel into the hole formed in one of the first or second bone, the rescue dowel comprising a tubular body sized and configured to be received by and frictionally engage with said hole, the tubular body defining a lumen therethrough; positioning a counter-rotation ring around the rescue dowel, wherein the counter-rotation ring comprises an annular body having a first surface and an opposing second surface, wherein a plurality of axial spikes extends from at least one of the first and second surfaces; inserting at least one axial spike into the first or second bone from which the rescue dowel is first inserted such that the first surface of the annular body abuts a surface that surrounds the rescue dowel; placing the first and second bones in an assembled relationship such that the hole of the other of the first or second bone receives the rescue dowel; inserting at least one axial spike into said other of the first or second bone such that the second surface of the annular body abuts a surface that surrounds the hole of said other of the first or second bone; wherein the counter-rotation ring rotationally fixes the first bone and second bone, thereby facilitating fusion of the assembled first and second bones.

Example 61: The method for straightening an interphalangeal joint according to any example herein, particularly example 60, further comprising defining channels through at least portions of the lengths of both the first and second bones, wherein the channels are sized and configured to receive a guide pin therethrough.

Example 62: The method for straightening an interphalangeal joint according to any example herein, particularly example 61, further comprising introducing the guide pin into the channel of the first bone.

Example 63: The method for straightening an interphalangeal joint according to any example herein, particularly examples 61 or 62, further comprising introducing the guide pin into the channel of the second bone.

Example 64: The method for straightening an interphalangeal joint according to any example herein, particularly examples 60 or 63, wherein the step of removing material from one of the first or second bone so as to form a hole further comprises engaging the guide pin with a central lumen of a hole reamer.

Example 65: The method for straightening an interphalangeal joint according to any example herein, particularly example 64, wherein the step of placing the first and second bones in an assembled relationship such that the hole of the other of the first or second bone receives the rescue dowel further comprises engaging the guide pin with the lumen of the rescue dowel.

Example 66: The method for straightening an interphalangeal joint according to any example herein, particularly example 65, wherein the step of removing material from the other of the first or second bone so as to form an opposing hole further comprises engaging the guide pin with a central lumen of a hole reamer.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

Although the description provides a specific order of method steps, the order of the steps may differ from what is described. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

Additional advantages may be learned by practice. The advantages will be realized and attained by means of the elements and combinations particularly pointed out in the appended claims. It is to be understood that the description and are exemplary and explanatory only and are not restrictive, as claimed.

What is claimed:

1. An interphalangeal joint fusion system comprising:
a counter-rotation ring comprising an annular body including a proximal spike extending axially from a proximal surface of the annular body and a distal spike extending axially from a distal surface of the annular body.

2. The system of claim 1, wherein the proximal spike includes a plurality of proximal spikes extending axially from the proximal surface of the annular body and the distal spike includes a plurality of distal spikes extending axially from the distal surface of the annular body.

3. The system of claim 2, wherein the plurality of proximal spikes are arranged circumferentially about the proximal surface of the annular body, and the plurality of distal spikes are arranged circumferentially about the distal surface of the annular body.

4. The system of claim 1, wherein at least one of the proximal spike or the distal spike has a decreasing width from the corresponding proximal surface or distal surface of the annular body toward an axial spike tip of the proximal spike or the distal spike.

5. The system of claim 1, wherein at least one of the proximal spike or distal spike includes an inner surface that is angled radially inward toward a central axis of the annular body.

6. The system of claim 5, wherein at least one of the proximal spike or distal spike includes a barb extending radially inward from an inner surface of the spike toward a central axis of the annular body.

7. The system of claim 1, wherein at least one of the proximal spike or distal spike includes an inner surface extending generally parallel with a central axis of the annular body, and an outer surface having a decreasing taper from the corresponding proximal surface or distal surface of the annular body toward a spike tip of the proximal spike or distal spike.

8. The system of claim 1, wherein at least one of the proximal spike or distal spike includes a barb extending radially outward from an outer surface of the spike away from a central axis of the annular body.

9. The system of claim 1, wherein the annular body includes a plurality of circumferential slots arranged circumferentially about the annular body and extending at least partially through the annular body.

10. The system of claim 1, wherein the annular body includes a suture opening extending through the annular body.

11. The system of claim 1, further including a peg reamer comprising:
a cannulated shaft defining a central lumen extending therethrough;
a cutting end provided at a first end of the cannulated shaft, the cutting end including a plurality of cutting arms extending from the cannulated shaft, each cutting arm includes a cutting edge extending along an inner surface of each cutting arm; and
a locking end provided at an opposing second end of the cannulated shaft from the cutting end.

12. The system of claim 11, wherein the central lumen of the peg reamer cannulated shaft defines a central axis of the peg reamer, where and each of the cutting edges are spaced apart from the central axis of the cannulated shaft,
wherein rotation of the plurality of cutting arms about a central axis of the peg reamer defines a peg cutout region therebetween.

13. The system of claim 11, wherein rotation of the plurality of cutting arms about a central axis of the peg reamer defines a peg cutout region therebetween,
wherein the peg cutout region has a first diameter adjacent a proximal end of the peg cutout region and a larger second diameter adjacent a distal end of the peg cutout region.

14. The of claim 13, wherein the cutting edges slope outward relative to a central axis of the peg reamer toward a distal end of the cutting arms, wherein the peg cutout region second diameter is larger than the peg cutout region first diameter such that the peg cutout region is tapered.

15. The system of claim 1, further including a hole reamer comprising:
a cannulated shaft defining a central lumen extending therethrough;
a cutting end provided at a first end of the cannulated shaft, the cutting end including a plurality of cutting edges extending along an outer surface of the cutting end, and a counterbore offset a distance from a distal end of the cutting end; and
a locking end provided at an opposing second end of the cannulated shaft from the cutting end.

16. The system of claim 15, wherein the central lumen of the hole reamer cannulated shaft defines a central axis of the hole reamer,
wherein rotation of the plurality of cutting edges about the hole reamer central axis defines a hole cutout region.

17. The system of claim 16, wherein the hole cutout region has a first diameter adjacent the counterbore and a second diameter adjacent the distal end of the hole reamer,
wherein the plurality of cutting edges slope inward relative to the central axis from the counterbore toward the distal end of the hole reamer such that the hole cutout region second diameter is larger than the hole cutout region first diameter and the hole cutout region is tapered toward the distal end.

18. The system of claim 1, further including a rescue dowel comprising:
a tubular body defining a central lumen extending therethrough, the tubular body having a proximal end defining a proximal end opening extending to the central lumen and a distal end defining a distal end opening extending to the central lumen,
wherein the proximal end and distal end are sized and configured to frictionally engage with the hole cutout region.

19. A method for straightening an interphalangeal joint, comprising:
accessing an interphalangeal joint;
separating first and second bones on opposite sides of the interphalangeal joint;
removing material from one of the first or second bone so as to form a peg and removing material from the other bone so as to form an opposing hole, wherein the peg is sized and configured to be received by and frictionally engage with the hole;
positioning a counter-rotation ring around the peg, wherein the counter-rotation ring comprises an annular body having a first surface and an opposing second surface, wherein a plurality of axial spikes extend from at least one of the first and second surfaces;
inserting at least one axial spike into the first or second bone from which the peg is formed such that the first surface of the annular body abuts a surface that surrounds the peg;
placing the first and second bones in an assembled relationship such that the hole receives the peg;
inserting at least one axial spike into the first or second bone from which the hole is formed such that the second surface of the annular body abuts a surface that surrounds the hole;
wherein the counter-rotation ring rotationally fixes the first bone and second bone, thereby facilitating fusion of the assembled first and second bones.

20. A method for straightening an interphalangeal joint, comprising:
accessing an interphalangeal joint;
separating bones on opposite sides of the interphalangeal joint, wherein a first bone is proximal relative to the joint and a second bone is distal relative to the joint;
removing material from one of the first or second bone so as to form a hole and removing material from the other bone so as to form an opposing hole;
inserting a rescue dowel into the hole formed in one of the first or second bone, the rescue dowel comprising a tubular body sized and configured to be received by and frictionally engage with said hole, the tubular body defining a lumen therethrough;
positioning a counter-rotation ring around the rescue dowel, wherein the counter-rotation ring comprises an annular body having a first surface and an opposing second surface, wherein a plurality of axial spikes extends from at least one of the first and second surfaces;
inserting at least one axial spike into the first or second bone from which the rescue dowel is first inserted such that the first surface of the annular body abuts a surface that surrounds the rescue dowel;
placing the first and second bones in an assembled relationship such that the hole of the other of the first or second bone receives the rescue dowel;
inserting at least one axial spike into said other of the first or second bone such that the second surface of the annular body abuts a surface that surrounds the hole of said other of the first or second bone;
wherein the counter-rotation ring rotationally fixes the first bone and second bone, thereby facilitating fusion of the assembled first and second bones.
